(19) United States
(12) Patent Application Publication
(10) Pub. No.: US 2025/0251282 A1
(43) Pub. Date: Aug. 7, 2025

(54) Methods For Implementing Standardised Time Domain Diffuse Optical Spectroscopy In Wearables/Portables

(71) Applicant: Biopixs Ltd, Cork (IE)

(72) Inventors: Sanathana Konugolu Venkata Sekar, Cork (IE); Claudia Nunzia Guadagno, Milano (IT)

(21) Appl. No.: 18/856,051

(22) PCT Filed: Mar. 23, 2023
(86) PCT No.: PCT/IB2023/052857

§ 371 Date: Oct. 10, 2024

(30) Foreign Application Priority Data
Apr. 12, 2022 (GB) 2205454.8

(51) Int. Cl. G01J 3/02; A61B 5/00; A61B 5/1455; G01J 3/28; G01J 3/44

(52) U.S. Cl. CPC G01J 3/0297; A61B 5/0075; A61B 5/1455; G01J 3/2889; G01J 3/4412

(57) ABSTRACT

Disclosed is a wearable device configured to perform standardised time domain diffuse optical spectroscopy, comprising an illumination system with a light source configured to illuminate a target region of the body, a photodetection system configured to detect scattered light exiting the target region and provide a measurement signal, and a processing circuit for time of flight (ToF) data acquisition. At least a portion of the beam is coupled to the photodetection system via a first reference optical path comprising a tissue mimicking phantom with one or more known optical properties to provide a first reference signal. The processing circuit measures a ToF distribution for photons detected from the target region and determines one or more optical properties of the target region from measured ToF distribution; measures a ToF distribution for photons detected from the phantom and determines one or more calibration factors for use in determining the one or more optical properties of the target region; and determines one or more optical biomarker values based, at least in part, on the one or more optical properties of the target region.

### TECHNICAL FIELD

This invention relates generally to portable/wearable devices that perform optical measurements of physiological properties of the body for healthcare/biomedical applications, particularly methods of implementing standardised time domain diffuse optical spectroscopy therein.

### BACKGROUND TO THE INVENTION

The past two decades have seen great advances in the field of biophotonics, with numerous devices being developed that exploit the high penetration depth and low attenuation of light in biological tissues in the visible to near infrared (NIR) wavelength range (600-1100 nm) for medical and healthcare applications including, but not limited to, monitoring heart rate, respiration rate, and blood/tissue oxygen level (oximeters). Improved data analysis methods and continued miniaturisation of light sources and detectors in this wavelength range, as well as associated processing circuitry, has paved the way for the development of not only clinical devices, but also personal wearable biophotonic devices that are now commonplace in the sports/performance/lifestyle and healthcare market, such as smart watches, smart bands, and many other types, with various health monitoring functionalities.

Biological tissues are diffusive in nature. When light in the NIR spectral window interacts with tissue, it undergo scattering and absorption dependent on the presence and concentration of certain tissue constituents, known as biomarkers, e.g. haemoglobin variants such as oxy- and dehaemoglobin, oxygen saturation (arterial, venous and tissue), water, lipids, collagen, melanin, bilirubin, glucose, etc. The above mentioned biophotonic devices, and many more, work by determined one or more optical properties of biological tissue, i.e. absorption coefficient μₐ and reduced scattering coefficient μₛ′, from measurements of scattered light re-emitted from the tissue (known as diffuse optical spectroscopy), and estimating various biological/physiological properties of tissue (static or dynamic) based on the measured optical properties.

Almost all wearable biophotonics devices available today use continuous wave (CW) diffuse optical spectroscopy (DOS) technology, which is based on illumination with CW light sources and measurements of light attenuation. The widespread adoption of CW technology in the wearable biophotonic device market is largely due to the relatively simple and inexpensive instrumentation, coupled with the high speed and signal-to-noise ratio (SNR) of the measurement. However, CW-based devices have a number of disadvantages that limit their accuracy and reliability in normal use. In particular, measurements taken with CW instruments cannot provide separate quantitative estimates of absolute optical properties of tissue (μₐ and μₛ′), and are prone to significant errors from motion artefacts and superficial layer effects such as skin pigmentation. For this reason, CW technology is most effective at monitoring relative changes in biomarker values, and consequently results from different CW-based devices are rarely comparable.

Absorption and scattering are inherently coupled in CW measurements of scattered light. μₐ and μₛ′ can be disentangled to some extent by performing spatially resolved CW measurements (detecting at multiple source-detector separations, d) or angle resolved CW measurements (detecting light at different scattering angles, α) as illustrated in FIGS. 1(a) and 1 (b). This increases the accuracy of the estimated biomarker values, but at the expense of increased size, cost and complexity of the instrumentation and on-board signal processing requirements. The accuracy of the estimated biomarker values can also be improved by performing measurements at multiple wavelengths with a priori knowledge of the biomarker spectra.

State of the art pulse oximeters perform CW measurements at two different wavelengths (one in the red and the other in the IR range) and can measure variations in oxy- and deoxyhaemoglobin allowing the determination of arterial blood oxygen saturation levels (SpO₂). Blood in arterial vessels pulses at a frequency of around 2 Hz, while blood flux in venous and capillary vessels can be considered static, providing both pulsatile (AC) and stationary (DC) signal components at the detector. Scattering variability between different subjects is accounted for by taking the ratio of AC to DC signal components, which is related to the scattering contribution of the tissue. This reduces distortion from inter-subject scattering variability, but there is no active solution for compensating for distortion arising from the skin pigmentation or motion.

Superficial layers in or on the surface of the tissue, such as melanocytes in the epidermis that are responsible for skin pigmentation, attenuate the light and introduce errors into the CW measurements of optical properties. In fact, skin colour related distortion in CW measurements is one of the biggest challenges currently faced in commercial biophotonic medical/healthcare devices such as pulse oximeters: melanocytes in the epidermis absorb light differently depending on their specific colour, meaning CW-based measurements of biomarker values can vary from subject to subject due to skin colour variation, even if the actual optical properties of the tissue are the same. For example, during the COVID-19 pandemic, pulse oximeters and their overestimation of blood oxygen (SpO₂) levels in the darkly pigmented tissues is blamed for failing to detect hypoxia in the dark-skinned population, such as Africans (see “2021.01.25 Letter to FDA re bias in pulse oximetry measurements” www.warren.senate.gov, and M. W. Sjoding et al. “Racial Bias in Pulse Oximetry Measurement” in New England Journal of Medicine, Vol. 383, 25 pp 2477-2478 (2020)).

Motion artefacts pose a challenge in all wearable biophotonic devices (e.g. fitness trackers, finger pulse oximeters, smart watches, etc.) where movement of the subject is common. Motion causes (primarily) transient decoupling of the wearable device from the body allowing direct light from the light source or ambient light from the surrounding environment to reach the detector and/or modulation of signal due to motion of tissue itself. Motion artefacts are especially problematic in CW measurements of light intensity, in particular, adding noise and error to photoplethysmography (PPG) signals used in pulse oximeters. While CW-based devices can be configured to detect, to some extent, extreme movement by analysing spurious spikes in the detected signal, they are not effective at detecting signal artefacts from smaller movements which can still cause significant errors in the extracted optical properties.

The above-mentioned problems are not limited to oximeters, but are inherent to any type of CW-based wearable biophotonic device.

Absolute measurements of absorption and scattering properties can be obtained using time domain (TD) or frequency domain (FD) DOS instruments and can provide more accurate and reliable quantification of various physiological processes. For example, separate absolute measurements of μₐ and μₛ′ can provide absolute values of oxy- and deoxyhaemoglobin from which tissue oxygen saturation can be determined in a single measurement at a single source-detector separation d. Implementing such measurements in practical wearable devices would be a significant advancement in the field. However, despite the inherent advantages, TD and FD measurements are technically more complex and expensive than CW measurements, which has made absolute measurements of tissue optical properties a niche technology implemented almost exclusively in large scale laboratory set ups to date.

The TD technique is promising candidate for next generation wearable devices, because of its relative simplicity compared to FD techniques, and has been successfully used (in a lab set up) to measure tissue optical properties at different locations on the body, including the wrists, trochanter, calcaneus, thyroid and abdomen. However, traditional TD measurements suffer from relatively low photon count rates of a few million photons per second (pps) which limits the SNR, and slow data acquisition compared to the CW DOS technique. By contrast CW-based devices can typically detect nW of light, or around 10⁹-10¹² pps. This, together with the expensive and complex instrumentation, has made the implementation of TD DOS in commercial wearable devices impractical.

Low SNR and slow data acquisition is inherent to the way the TD measurement is traditionally implemented. The TD approach uses picosecond pulsed lasers and time-resolved single-photon detection to discriminate photons on the basis of their arrival time and measure a time of flight (ToF) distribution from which the optical properties (μₐ and μₛ′) can be extracted. The most common approach for measuring ToF is based on time correlated single photon counting (TCSPC). In TCSPC, it is necessary that the probability to detect more than one photon for each excitation pulse is negligible. As such, the ToF distribution takes time to build up. Further, when multiple photons reach the detector per cycle, the “dead time” of the single photon detection system (the minimum time interval of which two consecutive events can be detected) means that photons arriving at shorter delays (so called “early photons”) are preferentially detected and the ToF distribution shows an accumulation of counts at the first part of the curve, an unwanted effect called “pile-up”. Another common approach to measuring ToF is time-gated single photon detection, which avoids the pile up effect to a certain extent, but has other draw backs which limit the count rate to a similar number. In practice, the repetition rate of light source has a lower limit to achieve enough counts (signal) without pile up and an upper limit to avoid unwanted “wrapping” of the ToF distribution. In traditional TD DOS systems, the pile-up effect, dead time of the single photon detection system and repetition rate of the light source all combine to limit the photon count rate of a few million photons per second (pps), and the maximum repetition rate of around 100 MHz.

However, recent advances in detector technology, based on ToF Lidars with parallel detection schemes developed for the automotive and consumer mobile industries, mean that next generation TD systems have the potential to reach photon count rates of 10¹¹ or higher without pile-up, paving the way for practical TD-based wearable devices that can compete with state of the art CW technologies (see F. Villa et al. “SPADs and SiPMs Arrays for Long-Range High-Speed Light Detection and Ranging (LiDAR)”, Sensors 21, 3839 (2021)).

Kernel™ has demonstrated a head-mounted TD-based biophotonic device for measuring brain neural activity via TCSPC implementing a parallel detector system that provides high photon count rates of 1 Gops without pile up, and at a sampling frequency of 200 Hz for fast acquisition (see Han Y. Ban et al. “Kernel Flow: A high count channel count scalable TD-fNIRS system”, Integrated Sensors for Biological and Neural Imaging, Proceedings of SPIE Vol 11663 (2021)). US2019/0378869A1, US2020/0352445A1 and US2021/0352445A1 also disclose head-mountable TD DOS systems with multiple single photon detectors connected in parallel for measuring brain neural activity via TCSPC.

Despite the recent advances in ToF detector technology, TD-based wearable biophotonic devices remain a nascent technology, and many of the problems identified in CW-based wearable devices (some of which are discussed above) remain to be addressed. As such, there is a need to develop standardised methodologies for implementing TD DOS in next generation wearable devices that can provide more robust and accurate measurements of physiological parameters.

Aspects and embodiments of the present invention have been devised with the foregoing in mind.

### SUMMARY OF THE INVENTION

According to a first aspect of the invention, there is provided a portable or wearable device for biophotonic applications configured to perform standardised time domain diffuse optical spectroscopy. The device comprises an optical measurement system for performing time domain diffuse optical spectroscopy. The optical measurement system comprises an illumination system and a photodetection system. The illumination system comprises a light source configured to illuminate a target region of the body, at an injection point, with a pulsed or modulated beam of light thereby producing scattered light from the interaction of the light beam with the target region. The photodetection system is configured to detect scattered light exiting the target region at a distance from the injection point. The target region may comprise biological tissue, such as skin. The photodetection system may provide or output a measurement signal or output signal. The optical measurement system may further comprise a processing circuit in communication with the illumination system and the photodetection system for time of flight (ToF) or temporal data acquisition. The processing circuit may be configured to control or operate the illumination system and the photodetection system. The processing circuit may be configured to measure or generate a time of flight distribution of photons detected in response to the light beam being directed towards the target region, based at least in part on the measurement/output signal. The ToF distribution may represent histogram of photons detected during a measurement period. The processing circuit may further be configured to determine one or more optical properties of the target region from the measured ToF distribution, and determine one or more optical biomarker values based, at least in part, on the one or more optical properties of the target region.

The one or more optical properties may include an absorption coefficient and/or a reduced scattering coefficient. The one or more optical properties may be extracted from the shape of the ToF distribution, e.g. based on a comparison to, or fitting with, a model ToF distribution derived from a theoretical diffusive media model convolved with the instrument response function (IRF).

The optical measurement system is configured to measure the instrument response function (IRF) and/or one or more optical properties of a tissue mimicking phantom to compensate for drift/distortion in the light source/detector and/or to calibrate and monitor device performance over time. The IRF can be used in extracting optical properties from the ToF data and/or to determine an overall attenuation by the target region for use in extracting superficial layer properties (see below).

The optical measurement system comprises a first reference optical path between the light source and photodetection system to couple at least a portion of the light beam to the photodetection system to provide a first reference signal, wherein the first reference optical path comprises a tissue mimicking phantom with one or more known optical properties. In this case, the processing circuit may further be configured to: measure a ToF distribution for photons detected in response to the at least a portion of the light beam being directed towards the phantom along the first reference optical path; extract one or more optical properties of the phantom from the shape of the measured ToF distribution.

The processing circuit may further be configured to determine, based on a comparison of the extracted one or more optical properties with the known optical properties of the phantom, a first calibration factor to apply to the one or more optical properties extracted from the ToF distribution measured from the target region.

The processing circuit may be configured to extract the IRF from the measured ToF distribution for detected photons from the phantom based on the known optical properties of the phantom (i.e. by back calculating).

Alternatively or additionally, the optical measurement system may use a separate reference optical path to measure the IRF. In this case, the optical measurement system comprises a second reference optical path between the light source and photodetection system to couple at least a portion of the light beam directly to the photodetection system and provide a second reference signal. The processing circuit may be configured to measure the IRF for photons detected in response to the at least a portion of the light beam being directed towards the photodetection system along the second reference optical path. The first reference optical path may be omitted if only IRF measurements are required.

The processing circuit may be configured to store the measured/extracted IRF (obtained from either reference optical path) for use in extracting the one or more optical properties of the target region from the shape of the ToF distribution, and/or for compensating for drift/distortion in the light source/detector.

The processing circuit may be configured to extract the one or more optical properties of the target region from the shape of the measured ToF distribution of photons from the target region based on a comparison or fit to a model ToF distribution derived from a theoretical diffusive media model convolved with the stored IRF. This may compensate for any temporal drift in the light source output.

The processing circuit may be configured to extract the one or more optical properties of the phantom from the shape of the measured ToF distribution by comparison to a model ToF distribution derived from a theoretical diffusive media model convolved with the stored IRF.

The processing circuit may be configured to measure and use the stored IRF to compensate for temporal drift in the optical measurement system in real time. Using the measured IRF to generate the model ToF curve accounts for drift in the pulse or waveform sequence. The processing circuit may be configured to determine, based on the stored IRF, a second calibration factor to correct for drift in the optical measurement system (e.g. drift in the light source power output, detector efficiency, other drifts etc.). The second calibration factor may be used to determine an overall attenuation of light by the target region (described further below).

The processing circuit may be configured to measure and update the stored IRF periodically and/or in response to a user input. The rate of the IRF acquisition may be dependent on whether it is used in determining static or dynamic optical properties.

The processing circuit may be configured to store the measured first and/or second calibration factor. The processing circuit may be configured to measure and update the stored first and/or second calibration factor periodically and/or in response to a user input.

The optical measurement system may be configured to operate in a measurement mode in which the ToF distribution of photons detected from the target region is measured, a first reference mode in which the ToF distribution for photons detected from the phantom is measured and/or a second reference mode in which the IRF is measured.

The ToF distribution of photons detected from the phantom and/or the IRF (measured using the first or second reference optical path) may be measured in sequence with the ToF distribution of photons detects from the target region, by selectively coupling the at least a portion of the light beam to the photodetection via the first and/or second reference optical path. Alternatively or additionally, the first and/or second optical reference path may include a delay line to temporally separate the respective first and/or second reference signal from the measurement signal and permit simultaneous/parallel measurement of the ToF distribution of photons detected from the target region, and the IRF and/or the ToF distribution of photons detected from the phantom.

The optical measurement system may comprise a first optical element/component operable (in response a control signal) to selectively couple the at least a portion of the light beam to the photodetection system, or allow the at least a portion of the light beam to reach the photodetection system, via the first reference optical path to measure the ToF distribution of photons detected from the phantom (in the first reference mode).

The optical measurement system may comprise a second optical element/component operable (in response a control signal) to selectively couple the at least a portion of the light beam to the photodetection system, or allow the at least a portion of the light beam to reach the photodetection system, via the second reference optical path to measure the IRF (in the second reference mode).

The processing circuit may be configured to send a control signal to the first optical element and/or the second optical element to change a state of the respective optical element (and thereby switch between the measurement mode and the first or second reference mode).

The first and/or second optical element/component may be operable to selectively block, transmit or direct the at least a portion of the beam along the first reference optical path. The first and/or second optical element may be or comprise one or more of: a moveable mirror, movable beam splitter, a moveable attenuator, adjustable attenuator, shutter, optical filters, variable optical filters, liquid crystal device, and an electro-absorption modulator.

The first and/or second reference optical path may comprise an optical waveguide, optical fibre or light pipe for guiding the light pulses to the photodetection system.

The photodetection system may comprise a single photon detector and a time-to-digital converter (TDC) coupled to the single photon detector. The processing circuit may be configured to determine a ToF of photons detected at the single photon detector, at least in part, on the output of the TDC, and generate the ToF distribution representing the temporal distribution of photons detected by the single photon detector accumulated over a measurement period.

The light source may be or comprise a pulsed laser or light emitting diode (LED), or a modulated continuous wave (CW) laser or LED. A pulsed light source may output a temporally uniform sequence of pulses at a repetition rate. A modulated CW light source may output a temporally uniform or non-uniform sequence of pulses or waveforms. A temporally non-uniform sequence of pulses or waveforms may have a sequence repeat rate.

ToF may be defined as the time difference between the arrival time of a detected photon and the emission of light from the light source. The optical measurement system may be configured to perform direct or indirect ToF measurements.

Direct ToF involves illuminating the target region with a beam of light pulses having a temporally uniform pulse sequence, and measurement of photon arrival times with respect to a light pulse that directly produces a ToF distribution that can be compared with a model ToF distribution to extract the optical properties. In the context of this disclosure, direct ToF measurements produce direct ToF data/information and a direct ToF distribution. Direct ToF requires picosecond light pulses (pulse width less than 1 ns) typically generated using a pulses laser or LED, high bandwidth detectors and timing electronics.

In direct ToF, the processing circuit may be configured to determine/measure a ToF of detected photons with respect to a light pulse based, at least in part, on the output of the photodetection system; and generate a ToF distribution of photons detected by the photodetection system accumulated over a plurality of light pulses. ToF measurements produce ToF data.

Direct ToF measurements can be performed using time correlated single photon counting (TCSPC), by time gated single photon detection, a combination of time gating and TCSPC, or by fast sampling of the single photon detector output using an analogue to digital converter (ADC).

For TCSPC, the photodetection system includes a time to digital converter (TDC) coupled to the output of a single photon detector to output a signal containing information on the ToF that can be used to generate the direct ToF distribution for detected photons. The TDC may receive a timing signal from the light source representing emission of light pulse. In other words, the TDC time stamps the individual detected photons.

For time gating, the photodetection system includes a gated single photon photodetector configured to measure a signal intensity within a predefined time gate window. The time gate window is associated with a gate delay time (defined with respect to the emission of a light pulse) that is progressively scanned/stepped across a delay time window of interest that includes the ToF distribution. As such, no TDC is required. However, a global clock keeps the timing resolution of the gate position, start and stop timing.

The TCSPC approach is sensitive to all photons reaching the photodetector regardless of the ToF, whereas the time gating approach is sensitive only to photons detected within the time gate window. Time gating has the advantage of time filtering the detected scattered light, and reducing distortions from ambient light, but has reduced detection efficiency, and takes longer to acquire the ToF distribution. On the other hand, TCSPC is limited by the dead time of the single photon detector and TDC, pile up from a limited laser repetition rate.

A TDC can be used in conjunction with time gating to provide increased time resolution, by measuring ToF of individual photons detected within the time gate window.

Indirect ToF measurements involve illuminating the target region with a temporally non-uniform pulse or waveform sequence, which may also be referred to as an indirect ToF input function (i.e. an amplitude or frequency modulated light beam). The detection arrangement is the same as direct ToF measurements, but indirect ToF measurements produce ToF data/information that is modulated by the indirect ToF input function (indirect ToF data). Direct ToF information can be recovered or extracted by applying one or more processing steps. Indirect ToF measurements may use modulated CW light source or a pulsed light source. The output of a CW light source is modulated to provide a temporally non-uniform pulse sequence or waveform sequence. The temporally non-uniform pulse sequence or waveform sequence can vary depending on kind of indirect ToF method used e.g., frequency domain ToF (uses a sequence of sinusoidally modulated signals with range of different frequencies around 40-300 MHz), spread spectrum (uses a pseudorandom sequence of pulses with minimum width similar to the resolution needed in the ToF distribution, e.g. 10-100 ps), and other transform methods. Compared to direct ToF measurements, indirect ToF uses a high duty cycle and low peak power light beam (modulated CW light source) which allows it to be exploited to gain both high count rate ToF and CW information (CW information can be obtained by integrating the ToF data/information).

Indirect ToF data may be referred to as being in indirect ToF space, and direct ToF data may be referred to as being direct ToF space. The processing circuit may be configured to process indirect ToF modulated information from the target region to extract/generate a direct ToF distribution, and then extract optical parameters from the direct ToF distribution based on a fit to a model ToF distribution (a theoretical model convolved with the IRF). Alternatively, the processing circuit may be configured to extract optical properties from the indirect ToF data by transforming the model ToF distribution into an indirect model ToF distribution (i.e. transforming into indirect ToF space) and fitting it to the measured indirect ToF data (depending on the signal to noise ratio and dynamic range).

The processing circuit may be configured to measure a series of ToF distributions over a period of time to monitor the one or more optical properties and/or optical biomarker values. The one or more optical properties may be static or dynamic (i.e. changing with time) optical properties. The extracted one or more optical properties may be associated with the bulk of the target region, e.g. deep tissue, and/or a superficial layer of the target region.

The photodetection system may be a high count rate photodetection system. The high count rate photodetection system may have a photon count rate greater than 100 M or 1 G counts per second without pile up. The photodetection system may comprise an array of single photon detectors. In this case, the processing circuit may be configured to: determine a ToF of photons detected at each single photon detector based, at least in part, on the outputs of the plurality of single photon detectors; and generate a ToF distribution of the sum of photons detected by the array of single photon detectors accumulated over a measurement period. Each single photon detector in the array may be referred to as a pixel photodetector.

Combining the counts/output from each detector of the array, while each avoiding pile up, provides a photodetection system with an overall high count rate without pile up. In this case, the optical measurement system may be configured to provide a photon count rate of greater than 100 M counts per second (cps), or greater than 1 Gcps without pile up (i.e. overall count rate summed over all detectors in the array).

The active area of the array of single photon detectors may be configured to provide a desired photon count rate. For example, for individual detectors with a given active area, the more detectors in the array, the greater the overall photon count rate without pile up.

The photodetection system may further comprise a plurality of time-to-digital converters (TDC), wherein each TDC is coupled to a respective one or group of single photon detectors in the array. The outputs of each TDC may be coupled together. In this case, the processing circuit may be configured to determine a ToF of photons detected by the array of single photon detectors based, at least in part, on the combined output of the plurality of TDCs.

Where the processing circuit determines the ToF of photons by time gated single photon detection, the system may or may not include TDCs. For example, for time gating, the single photon detector array may be operated in free running mode, and instead of having individual TDCs, the processing circuit may include a global time gating circuit which samples the output of the photodetector array at a particular time delay with a particular temporal resolution (i.e. a time gate with a certain width). Photons arriving at the particular time gate will be counted by the processing circuit and this process is repeated sequentially as the time gate moves across the time window of interest.

The array of single photon detectors may be divided into a plurality of (spatial) segments each comprising a subgroup of single photon detectors (pixels). A subgroup may comprise at least two single photon detectors. The outputs of the or each TDC associated with a respective segment or subgroup may be coupled together. This may provide separate ToF signals. The optical measurement system may be configured to couple scattered light exiting the target region to a first segment (of the plurality of segments) of the array to provide the measurement signal, and to couple the at least a portion of the light beam directed along the first reference optical path comprising the tissue mimicking phantom to a second segment of the array to provide the first reference signal. Optionally or preferably, the optical measurement system may be configured to measure the measurement signal and first reference signal simultaneously.

The processing circuit may be configured to determine a ToF of photons detected at each single photon detector in a respective segment based, at least in part, on the coupled output of the or each TDC associated with that segment; and generate, for each segment, a ToF distribution of the sum of photons detected by the subgroup of single photon detectors in the respective segment accumulated over a plurality of light pulses. The processing circuit may be configured to determine a ToF of photons by time correlated single photon counting, or by time gated single photon detection. Optionally or preferably, the optical measurement system may be configured to provide a photon count rate of greater than 100 Mcps without pile up.

Where the second reference arm is present, the optical measurement system may be configured to couple the at least a portion of the light beam directed along the second reference optical path to a third segment of the array to provide the second reference signal. Optionally or preferably, the optical measurement system may be configured to measure the measurement signal and first and/or second reference signal simultaneously.

The optical measurement system can be configured to control the source power and efficiency of the optical measurement system in one of the following ways: 1) by changing the repetition rate or duty cycle of the pulse sequence or by introducing delays between two successive sequences; 2) by varying the optical power of the light beam by introducing a variable optical filter in the illumination between and/or by adjusting the source power using a source driver.

The illumination system and/or the photodetection system may comprise one or more optical filters and/or variable optical filters to tune wavelength of the light beam or detected photons, control the optical power on the target region, and/or to minimise ambient light contamination in the measurement signal. The optical filter(s) may be or comprise one or more of: a bandpass, multi-bandpass, long pass, short pass, or neutral density filter. The one or more optical filters or variable optical filters may be positioned at the light source and/or detector. In one example, a bandpass filter may be placed on the detector that allows only selected wavelengths (e.g. the wavelength of the light beam).

The optical measurement system may be configured to perform time ToF measurements and also continuous wave (CW) measurements of scattered light. The optical measurement system may be configured in a ToF measurement mode for ToF measurements of scattered light, and a CW measurement mode for CW measurements of scattered light. The CW mode can be implemented in software by the processing circuit, and/or in hardware by the illumination system and photodetection system.

In software CW mode, the processing circuit is configured to extract CW data/information from the measured (direct or indirect) ToF distribution of detected photons. CW data may be extracted from the integrated (total) photon count of the ToF distribution which is equal to the amount of scattered light detected in the measurement period, representing CW information. The processing circuit may be configured to acquire both ToF data and CW data at each measurement period. Software CW mode may be implemented when using modulated or pulsed light, and with direct or indirect ToF measurements.

To implement a hardware CW mode, the light source may be or comprise a first light source configured to operate in pulsed/modulated mode or continuous wave (CW) mode. The processing circuit may be configured to operate the first light source in a ToF mode and a continuous wave (CW) mode. In the TOF mode, the first light source outputs a beam of light pulses for direct ToF measurements of scattered light, or a beam of modulated light with a temporally non-uniform waveform sequence for indirect ToF measurements. In the CW mode, the first light source outputs a continuous beam of light for measurements of intensity of scattered light.

Alternatively or additionally, the illumination system may further comprise a dedicated CW light source. The illumination system may further comprise second light source in communication with the processing circuit and configured to illuminate the target region of the body with a continuous beam of light. Optionally or preferably, the second light source may be co-located with the first light source e.g. so as to illuminate the target region at substantially the same injection point.

To implement the hardware CW mode, the photodetection system may comprise a first photodetector configured to operate in ToF (single photon detection) mode and continuous wave (CW) mode. The processing circuit may be configured to operate the first photodetector in a ToF mode and a continuous wave (CW). In the TOF mode, the first photodetector operates in a single photon detection mode for measuring a ToF distribution of detected photons detected in response to a pulsed or modulated light beam being directed to the target region. In ToF mode, the first photodetector may be operated in “Geiger mode”, biased beyond its reverse break down voltage. In the CW mode, the photodetector operates in a photodiode mode or photovoltaic mode or cumulative single photon counting mode for measuring an amount of scattered light in response to a continuous beam of light.

Additionally or alternatively, the photodetection system may further comprise a dedicated CW detector. The photodetection system may comprise a second photodetector in communication with the processing circuit, the second photodetector arranged at a distance from the first light source and/or the second light source to detect scattered light from the interaction of the continuous beam of light with the target region. Optionally or preferably, the second photodetector may be co-located with the first photodetector.

The first photodetector may be or comprise a ToF photodetector, single photon avalanche photodiode (SPAD), array of SPADs (e.g. a silicon photomultiplier (SiPM), or any other high time resolution/wide area detection technology.

The second photodetector may be or comprise a CW photodetector, e.g. a photodiode, or avalanche photodiode, SPAD, array of SPADs (e.g. a SiPM), high count rate ToF detector operating in cumulative single photon mode or any other high count rate/high power signal detector technology.

The processing circuit may be configured to dynamically switch the optical measurement system between a TOF measurement mode for ToF measurements of scattered light, and a (hardware or software) CW measurement mode for CW measurements of scattered light.

In the software CW measurement mode, the processing circuit is configured to measure a series of (direct or indirect) ToF distributions in response to the pulsed or modulated beam of light being directed to the target region, extract a timecourse of CW data (the amount of scattered light detected from the target region at each measurement period) from the series of ToF distributions; and determine one or more optical biomarker values based, at least in part, on one or more optical properties of the target region extracted from the measured timecourse.

Software CW mode may be particularly advantageous for indirect ToF measurements which can be implemented using modulated CW light sources which are cheaper, smaller and consume less power than pulsed light sources. By using a high duty cycle temporally nonuniform pulse/waveform sequence high average powers can be achieved suitable for high count rate CW measurements (e.g. which can we used for CW oximeter), whereas the measured indirect ToF distribution can be used derive calibration and corrections to compensate for scattering, superficial layer and motion artefacts (see below).

In the hardware CW measurement mode, the processing circuit is configured to: operate the first light source in CW mode, or operate the second light source, to output a continuous beam of light; operate the first photodetector in CW mode, or operate the second photodetector, to detect an amount of scattered light; measure a timecourse of the amount of scattered light detected from the target region in response to the continuous beam of light (i.e. a timecourse of CW data); and determine one or more optical biomarker values based, at least in part, on one or more optical properties of the target region extracted from the measured timecourse.

The processing circuit may further be configured to apply a correction factor to the one or more optical biomarker values determined in the (software or hardware) CW measurement mode, or process the CW data timecourse based, at least in part, on one or more static and/or dynamic optical properties or optical biomarker values determined in the ToF measurement mode. Optionally or preferably, the correction factor may be derived from an optical property of the target region or an optical property/optical biomarker value of a superficial layer of the target region determined in the ToF mode.

The correction factor may compensate for a scattering contribution to the optical properties estimated in CW mode, e.g. as this varies among different subjects. For example, the correction factor may be derived from reduced scattering coefficient μₛ′ measured in ToF mode may be used to compensate for or eliminate the scattering contribution to optical properties estimated from CW measurements (where μₐ and μₛ′ inherently coupled).

Additionally or alternatively, the correction factor may compensate for superficial layer distortion to optical properties/biomarker values estimated in (software of hardware) CW mode. The correction factor may be derived from the absorption coefficient of the superficial layer determined in ToF mode. This may improve any CW based biomarker assessment.

Additionally or alternatively, dynamic optical properties detected in ToF mode, e.g. changes in the superficial layer properties or shape of the rising portion of the ToF distribution due to system or target region motion, can be used to compensate for motion artefacts in CW measurements of light. For example, the processing circuit may be configured to process the measured timecourse of CW data to filter out or remove motion artefacts based on the dynamic optical properties detected in ToF mode.

Where the device is or comprises a pulse oximeter, a correction factor may be used to compensate arterial oxygen saturation (SpO2) with a scattering contribution and/or superficial layer contamination (see below), and/or motion artefact contribution (see below), determined from ToF measurements, and/or to complement the SpO2 value with tissue oxygen saturation (StO2).

Where the wearable device is configured to measure PPG signal or other time varying signals (e.g. brain hemodynamics or other organ's physiological or pathological changes), a correction factor may be used to compensate the determined optical properties and/or biomarker for a scattering contribution, superficial layer contribution, and/or motion artefact contribution determined from ToF measurements, and/or to complement the optical properties and/or biomarker determined in CW mode with optical properties and/or biomarker determined in ToF mode.

In this way, the ToF measurements can be used to compensate for various errors associated with scattering, superficial layer effects and motion artefacts in CW measurements in real time.

The processing circuit may be configured to determine the presence of and/or one or more optical properties of a superficial layer of the target region based on an analysis of the attenuation and/or shape of the measured ToF distribution.

A superficial layer may be defined as any layer whose absorption does not affect the shape of the ToF distribution. For example, the superficial layer may include but is not limited to a skin pigmentation layer (e.g. melanocytes or tattoo in the epidermis), scar tissue, nail, fat, and/or collagen (e.g. from immune response to an implantable device). The one or more optical properties of a superficial layer may include attenuation, an absorption coefficient and/or a reduced scattering coefficient. The processing circuit may be configured to monitor the one or more optical properties of a superficial layer over time. The one or more optical properties of a superficial layer may static or dynamic (i.e. changing with time). Dynamic optical properties of the superficial layer may be used to detect motion (see below).

In this way, the device can quantify static and/or dynamic properties of a superficial layer, which can be used as an additional optical biomarker, and/or to detect and/or compensate for skin pigmentation or motion artefacts (see below).

The presence of and/or the one or more optical properties of a superficial layer of the target region may be determined from the attenuation of the measured ToF based on a comparison of the overall attenuation of photons by the target region with the effective attenuation.

The processing circuit may be configured to determine an overall attenuation of photons by the target region based on a comparison of the integrated (total) photon count of the measured ToF distribution and the output of the light source. The processing circuit may be configured to calibrate the measured ToF distribution for drift in the optical measurement system by applying a correction factor determined from the IRF. The output of the light source and detector sensitivity may be calibrated using the IRF/second calibration factor determined in the first and/or second reference modes.

The processing circuit may be configured to determine an effective attenuation of photons by the target region based on the one or more optical properties of the target region extracted from the shape of the measured ToF distribution (e.g. based on a comparison or fit to a model ToF distribution derived from a theoretical diffusive media model convolved with the IRF). The effective attenuation may be proportional to the geometric mean of the absorption and reduced scattering coefficients.

The presence of and/or the one or more optical properties of a superficial layer of the target region may be determined from the shape of the measured ToF distribution based on a comparison or fit to a model ToF distribution derived from a theoretical multi-layer diffusive media model convolved with the IRF. The processing circuit may be configured to calibrate the measured ToF distribution for drift in the optical measurement system by applying a correction factor determined from the IRF.

Analysis of the attenuation and shape of the ToF curve may enable absorption and reduced scattering coefficients of the superficial layer to be extracted as well as absorption and reduced scattering coefficients of the bulk of the target region.

The IRF may be measured using the first or second reference optical path. The calibration factor for the light source output used for determining overall attenuation from the ToF curve may be measured using the first or second reference optical paths.

The light source may be configured to output a beam of light pulses at a plurality of different wavelengths. Alternatively or additionally, the illumination system may be or comprise a plurality of light sources, each configured to output a beam of light pulses at a different wavelength. The processing circuit may be configured to control the light source(s) to illuminate the target region with a light beam of different ones of the plurality of wavelengths, or a plurality of light beams with a respective plurality of wavelengths. The processing circuit may be configured to measure, at each wavelength, a ToF distribution and extract one or more optical properties of the target region at each wavelength. One or more optical filters or variable optical filters (e.g. bandpass filters) can be used to select the wavelength for the ToF measurement. The plurality of light beams at different wavelengths can be multiplexed. In this case, the processing circuit may be configured to extract the ToF distribution at each wavelength by performing an un-mixing method and extract one or more optical properties of the target region at each wavelength.

The processing circuit may be configured to determine the presence of and/or one or more optical properties of a superficial layer of the target region at each wavelength, optionally or preferably, based on a comparison with one or more stored superficial layer absorption spectra. For example, a superficial layer may have a characteristic absorption spectrum, which enables it to be identified. Where there are multiple layers contributing to the superficial layer (e.g. melanin, nail polish etc.), each may have a different characteristic absorption spectrum.

The processing circuit may be configured to determine an optical biomarker value of the superficial layer based, at least in part, on the determined one or more optical properties of the superficial layer. The optical biomarker value of the superficial layer may be or comprise a superficial layer identifier/index and/or a skin pigment index. For example, the skin pigment index may indicate a skin colour or level of pigmentation. The superficial layer identifier/index may indicate what the superficial layer is, e.g. skin pigmentation, scare tissue, nail, nail polish, collagen, layer of blood etc.

Information on a superficial layer obtained from ToF measurements may be used, in conjunction with CW measurements (in software or hardware CW mode) described above, to compensate for superficial layer related errors and artefacts in the optical biomarkers estimated from CW measurements.

The processing circuit may be configured to measure a series of ToF distributions over a period of time, and detect a motion artefact in at least one of the ToF distributions based on dynamic changes in the shape and/or attenuation of the ToF distributions.

The motion artefact may be due to device motion relative to the skin/tissue, and/or motion of tissue (e.g. expansion and contraction). Information on a motion artefacts obtained from ToF measurements may be used, in conjunction with CW measurements (in software or hardware CW mode) described above, to compensate for motion artefacts in the optical biomarkers.

Motion artefacts may be detected based on dynamic changes in the determined one or more optical properties of the superficial layer due to motion. The processing circuit may be configured to determine, for each ToF distribution in the time series, one or more optical properties of a superficial layer of the target region based on an analysis of the attenuation and/or shape of the ToF distribution (e.g. as described above); and detect a motion artefact in at least one of the ToF distributions based on dynamic changes in the determined one or more optical properties of the superficial layer.

To capture motion artefacts, the processing circuit may be configured to measure the series of ToF at a rate of at least 5 Hz or at least 10 Hz, or at least 50 Hz. Motion artefacts typically add noise to measured PPG signals (obtained from CW measurements) which follow the heart rate, i.e. in the frequency range 1 to 3 Hz (higher rates during intense cardio exercise). As such, to resolve such artefacts, and to resolve features in the PPG signals such as early and late systolic peak, dicrotic notch, depending on the application a measurement/sampling frequency of at least few times (2-10) the highest frequency of interest is needed to satisfy the Nyquist criteria. However, higher rates may be needed while performing intense movement, e.g. intense movement of target region during running or athletic event. Slower motion artefacts, e.g. due to change in body position during sleep, can be monitored slowly in sub Hz (0.01-1 Hz) range.

Alternatively or additionally, the processing circuit may be configured to detect a motion artefact in at least one of the ToF distribution based on a comparison of the shape of the rising portion of the measured ToF distribution with a model ToF distribution derived from a theoretical diffusive media model (single or multi-layer) convolved with the IRF. The motion artefact may be detected based on a predefined divergence from the expected shape of the rising portion of the ToF distribution, such as an anomalous peak, dip and/or curvature.

In response to detecting a motion artefact, the processing circuit may be configured to extract one or more optical properties of the target region from the shape of the tail portion of the ToF distribution. Alternatively or additionally, the processing circuit may be configured to compare the shape of the rising portion of the ToF distribution with a database of simulated motion artefacts (e.g. curves generated under controlled motion conditions), apply a correction to the model ToF distribution based on a selected one of the simulated motion artefacts, and extract the one or more optical properties of the target region from the shape of the ToF distribution using the corrected model ToF distribution.

In this way, motion artefacts can be effectively detected and compensated for in real time, reducing errors in the extracted optical properties.

The motion artefact itself, e.g. detection of changes in dynamics of superficial layer, and/or early photons) can be used as optical biomarker or physiological monitor of the target region or human body or behaviour.

The wearable device may be configured to be attached to a wrist, chest, leg, arm, abdomen, muscle, neck, back or head.

The one or more optical biomarkers may be selected from the group including but not limited to: tissue oxygen saturation, arterial oxygen saturation, venous oxygen saturation, oxy-haemoglobin, total haemoglobin, methe-haemoglobin, glycated haemoglobin, carboxy haemoglobin, deoxy-haemoglobin, lipid, water, collagen, hydration, glucose, melanin, thyrosine, thyroglobulin, cytochrome c-oxidise.

The or each light source may be or comprise a laser or light emitting diode. The light source may be configured to output light in the wavelength range between 450 nm and 2400 nm, or between 650 nm and 1350 nm.

The photodetection system may comprise one or more single photon detectors and timing circuitry coupled to the single photon detector(s). The single photon detector(s) may be or comprise a single photon avalanche photodiode (SPAD), an array of SPADs, a time gated SPAD, an array of time gated SPADs, or a silicon photomultiplier (SiPM). The one or more single photon detector may be implemented in silicon photonics. Time gated SPADs may be software gated, hardware gated, or free running (ungated) with universal time gate enabling SPAD array output.

The timing circuitry may be or comprise one or more time to digital converters, and/or fast (high bandwidth) analogue amplifiers/circuits. Each TDC may be connected to one or a group of single photon detectors.

The wearable device may be or comprise a pulse oximeter. In this case, the determined one or more optical biomarker values include tissue oxygen saturation, arterial oxygen saturation, oxy-haemoglobin, total haemoglobin, methe-haemoglobin, glycated haemoglobin, carboxy haemoglobin and deoxy-haemoglobin.

The processing circuit comprise any suitable combination of components for controlling the photodetection system and light source, acquiring temporal data for detected photons, signal processing and storage, including but not limited to one or more of: timing electronics such as time-to-digital converters, gated ring oscillators, processors, memories, auto correlators, cross correlators, on-chip histogrammer, high bandwidth analogue amplifier/circuits, laser drivers, gain switch circuit, mode locking circuit, fast optical modulators e.g. EAM, silicon photonics integrated optical components e.g. EAM, waveguides, light source, drivers, processors, micro-optical components, filters, CMOS implemented source, detection and processing circuits.

According to second aspect of the present invention, there is provided a wearable pulse oximeter or PPG signal detector comprising the optical measurement system described in the first aspect. The wearable pulse oximeter or PPG signal detector may be configured to perform standardised indirect ToF measurements (e.g. spread spectrum or other indirect ToF method) and CW measurements in a software CW mode, as described in the first aspect.

The indirect ToF mode may provide scattering and/or superficial layer information. The software CW mode (sum of photons acquired using indirect ToF mode) provides a high resolution PPG signal which can be used, e.g. to determine arterial tissue oxygenation, and the optical properties extracted from the indirect ToF measurements provides tissue oxygen saturation. In addition, the device can be configured to perform ToF and CW measurements at multiple wavelengths to measure all forms of haemoglobin and other optical biomarkers.

According to a third aspect of the present invention, there is provided one or more methods of implementing standardised time domain diffuse optical spectroscopy in a wearable device as described in the first aspect, corresponding to one or more of the features of the wearable device of the first aspect.

According to a fourth aspect of the invention, there is provided a method of performing standardised time domain diffuse optical spectroscopy in a wearable device comprising an optical measurement system configured to perform time domain diffuse optical spectroscopy, the optical measurement system comprising an illumination system and a photodetection system. The method comprises measuring a (direct or indirect) ToF distribution for photons detected in response to a pulsed or modulated beam of light being directed towards a target region of the body. The method may further comprise measuring the IRF in response to a pulsed or modulated beam of light being directed towards a photodetection system along a reference optical path without interacting with the target region. The method may further comprise extracting one or more optical properties of the target region from the shape of the measured ToF distribution by comparison to a model ToF curve derived from a theoretical diffusive media model convolved with the measured IRF. Additionally or alternatively, the method may comprise determining, based on the measured IRF, a calibration factor to apply to the ToF distribution to correct for drift in the optical measurement system. The calibration factor may be used for determining an overall attenuation of light by the target region, e.g. for use in determining one or more optical properties of a superficial layer of the target region.

The method may further comprise measuring a ToF distribution for photons detected in response to a pulsed or modulated beam of light being directed towards a tissue mimicking phantom located in the same or a different reference optical path, and extracting one or more optical properties of the phantom from the shape of the measured ToF distribution by comparison to a model ToF curve derived from a theoretical diffusive media model convolved with the measured IRF. The method may further comprise determining, based on a comparison of the extracted one or more optical properties with known optical properties of the phantom, a calibration factor to apply to the one or more optical properties extracted from the shape of the ToF distribution measured from the target region.

The method may further comprise performing ToF measurements and CW measurements in the same wearable device.

The method may comprise performing continuous wave (CW) diffuse optical spectroscopy based on ToF data using a software CW mode. The method may further comprise extracting a CW measurement signal from the measured ToF distribution from the target region by summing all or a part of the measured ToF distribution from the target region, the CW signal representing an amount of scattered light detected. The method may further comprise measuring a series of ToF distributions and extracting a timecourse of the CW measurement signal in response to a pulsed or modulated beam of light being directed to the target region, The method may further comprise determining one or more optical biomarker values based, at least in part, on an optical property of the target region extracted from the measured timecourse. The method may further comprise applying a correction factor to the one or more optical biomarker values based, at least in part, on one or more static and/or dynamic optical properties extracted from the ToF measurements, optionally or preferably, wherein the correction factor is derived from an optical property of a superficial layer of the target region determined from the ToF measurements.

The step of measuring a ToF distribution for photons detected in response to a pulsed or modulated beam of light being directed towards a target region of the body may comprise measuring a direct/indirect ToF distribution for photons detected in response to a pulsed/modulated beam of light being directed towards a target region of the body using a photodetection system with a count rate greater than 100 M or 1 G counts per second.

The method may further comprise detecting the presence of and/or determining one or more optical properties of a superficial layer of the target region based on based the attenuation and/or shape of the measured ToF distribution. The method may further comprise measuring a series of ToF distributions over a period of time; and detecting a motion artefact in at least one of the ToF distributions based on dynamic changes in the determined one or more optical properties of the superficial layer, optionally or preferably, based on dynamic changes in the shape and/or attenuation of the ToF distributions.

The optical measurement system may further be configured to perform continuous wave (CW) diffuse optical spectroscopy in a hardware CW mode. In this case, the method may comprise operating the optical measurement system in a CW mode; measuring a timecourse of the amount of scattered light detected from the target region in response to a continuous beam of light being directed to the target region; and determining one or more optical biomarker values based, at least in part, on an optical property of the target region extracted from the measured timecourse. The method may further comprise applying a correction factor to the one or more optical biomarker values based, at least in part, on one or more static and/or dynamic optical properties determined in the ToF measurement mode, optionally or preferably, derived from an optical property of a superficial layer of the target region determined in the ToF measurement mode.

According to a fifth aspect, there is provided, a method of performing standardised time domain diffuse optical spectroscopy in a wearable device, comprising: performing time of flight (ToF) measurements of photons detected in response to a pulsed or modulated beam of light being directed towards a target region of the body using a high count rate photodetection system to generate ToF data; extracting one or more optical properties of the target region from the ToF data; and summing all or some of the photons detected in a measurement period to extract a high count rate photoplethysmography (PPG) signal. The method may further comprise; extracting one or more optical biomarker values from the PPG signal; and/or extracting one or more optical properties of a superficial layer of the target region based on the shape and/or attenuation of the ToF data. The method may further comprise correcting the one or more optical biomarker values extracted from the PPG signal based on the one or more optical properties of the superficial layer extracted from the ToF data.

Optionally, the method may further comprise detecting a motion artefact in the ToF data based on dynamic changes in the shape and/or attenuation of the ToF data, and processing the PPG signal to remove the detected artefact.

According to a sixth aspect, there is provided a method of calibrating the wearable device of the first aspect, comprising: measuring a series of ToF distributions over a period of time; moving the device to induce a motion artefact in at least one of the measured ToF distributions; detecting a motion artefact in at least one of the ToF distributions based on dynamic changes in the shape and/or attenuation of the ToF distributions; and calibrating the device based on detected motion artifacts. Calibrating the device may comprise creating or updating a look-up table (assessable by the processing circuit) with one or more extracted parameters of the motion artefact for use in correcting for motion artefacts. For example, the processing circuit may be configured to compare actual ToF measurements with look up data to assess the amount of motion and compensate for it. Optionally or preferably, wherein moving the device comprises lifting the device, or shaking the device. The one or more extracted parameters may be or comprise fit parameters of the ToF curve/distribution or a portion of the ToF curve/distribution (e.g. fitting range, initial optical properties etc.).

Features described in connection with the wearable device have corresponding features definable with respect to the method(s), and vice versa, and these embodiments are specifically envisaged.

Features which are described in the context of separate aspects and embodiments of the invention may be used together and/or be interchangeable. Similarly, where features are, for brevity, described in the context of a single embodiment, these may also be provided separately or in any suitable sub-combination.

### BRIEF DESCRIPTION OF DRAWINGS

In order that the invention can be well understood, embodiments will now be discussed by way of example only with reference to the accompanying drawings, in which:

FIGS. 1(a) and (b) shows schematic diagrams illustrating the concept of continuous wave measurements of scattered light;

FIG. 2 shows a schematic diagram of a wearable device;

FIG. 3 shows a schematic diagram of an array of photodetectors for high count rate time of flight (ToF) measurements in the device of FIG. 2;

FIG. 4 shows example measured ToF data fitted with a model ToF distribution along with the instrument response function;

FIG. 5 shows experimental absorption spectra of different optical biomarker extracted from ToF measurements;

FIG. 6 shows a schematic diagram of a wearable device according to an embodiment of the invention;

FIG. 7 illustrates temporally separated reference and measurement signals obtained in reference and measurement modes of the device;

FIG. 8 shows an example method of performing indirect ToF measurements at high repetition rates according to an embodiment of the invention;

FIGS. 9(a) to (c) show schematic diagrams of different source detector configurations of a hybrid ToF/CW wearable device according to an embodiment of the invention;

FIGS. 10(a) and 10 (b) show example ToF measurements of a phantom with a superficial layer and a table of the extracted optical properties;

FIG. 11 shows the effect of direct light and ambient light on ToF measurements due to motion; and

FIG. 12 shows example ToF measurements on a phantom under different motion conditions.

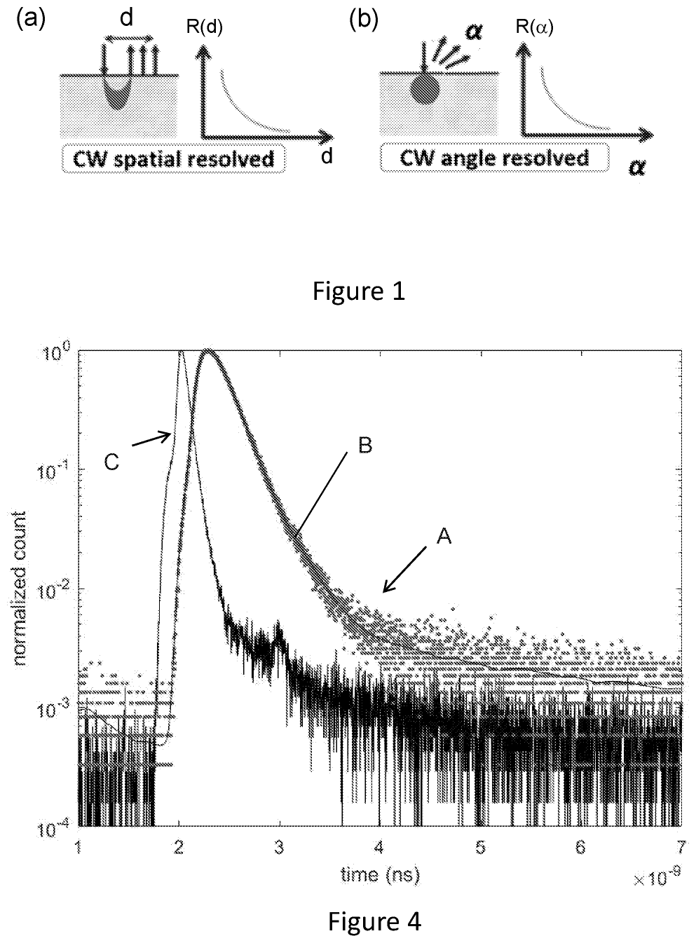

It should be noted that the figures are diagrammatic and may not be drawn to scale. Relative dimensions and proportions of parts of these figures may have been shown exaggerated or reduced in size, for the sake of clarity and convenience in the drawings. The same reference signs are generally used to refer to corresponding or similar features in modified and/or different embodiments.

### DETAILED DESCRIPTION

Diffuse optical spectroscopy (DOS) is a technique to extract optical properties (absorption and scattering) of diffusive media, such as biological tissue. As discussed in the background section, continuous wave (CW) based DOS has several problems, particularly when implemented in wearable devices, that limit the accuracy of the measurements, including superficial layer related distortions, motion artefacts, and the inability to separately evaluate and quantify the absorption coefficient μₐ and reduced scattering coefficient μₛ′ of the tissue. Aspects and embodiments of the present invention provide time domain (TD) based solutions to challenges faced with CW-based biophonic wearable devices.

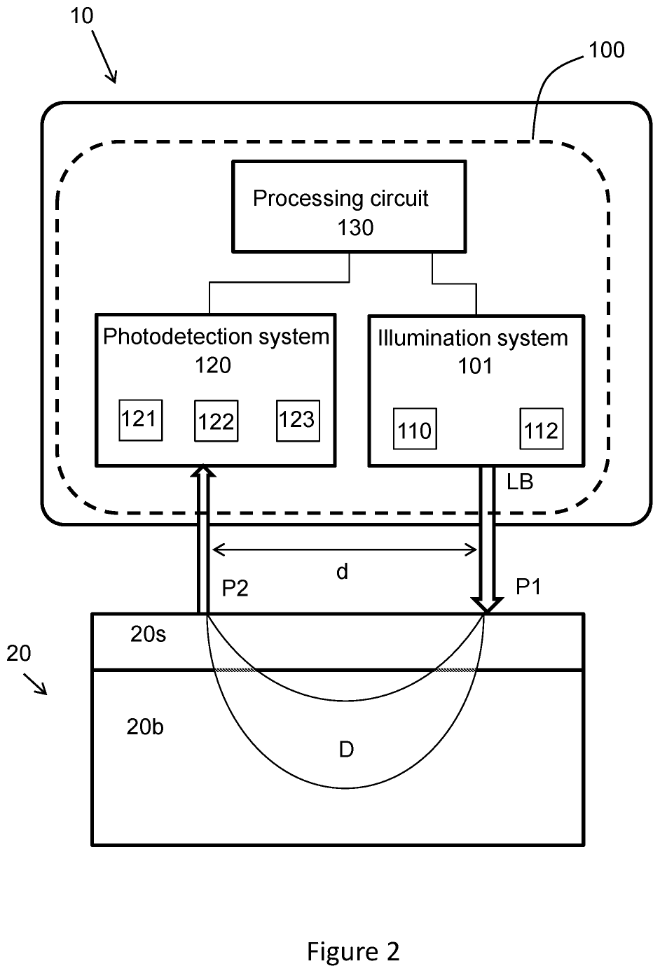

FIG. 2 shows a schematic diagram of a wearable device 10 comprising an optical measurement system 100 for performing TD DOS. The wearable device 10 may be configured to attach to any part of the body, e.g. a wrist, chest, leg, arm, abdomen, muscle, or head. For example, the wearable device 10 may comprise a strap or band (not shown) for attaching to the body.

The optical measurement system 100 comprises an illumination system 101, a photodetection system 120 and a processing circuit 130. The illumination system 101 comprises a light source 110 configured to illuminate a target region 20 of the body, at an injection point P1, with a beam of light pulses LB thereby producing scattered light from the interaction of the light pulses with the target region 20. The photodetection system 120 is configured to detect individual photons of the scattered light exiting the target region 20 at a distance d from the injection point P1. The distance d between the injection point P1 and the detection point P2 is typically in the range of 1 to 3 cm, as is known in the art. The target region 20 comprises biological tissue, including skin and underlying deep tissue. The target region 20 typically comprises a superficial layer 20s and a bulk layer 20b, as discussed in more detail below. In the illustrated embodiment, the device 10 is configured in reflection geometry. However, it will be appreciated that the device 10 can instead be configured in transmission geometry (not shown).

The photodetection system 120 comprises one or more single photon avalanche photodiodes (SPADs) 121 and timing circuitry 122 for sampling the output of the SPAD(s) 121. The light source 110 comprises pulsed laser or light emitting diode (LED), or a modulated laser/LED, operating in the visible to near infrared (NIR) wavelength range (450 nm to 2400 nm). Light emitted from the light source 110 may be directed to the target region 20 via an optical fibre (not shown). Further, scattered light exiting the target region 20 at the detection point P2 may be collected and directed to the photodetection system 120 via an optical fibre. Thus, the light source 110 and SPAD(s) 121 need not be located at the respective injection and detection points P1, P2. The illumination system 101 system and the photodetection system 120 comprise one or more optical filters/variable optical filters 112, 123 to tune wavelength of the light beam LB or detected photons, control the optical power on the target region 20, and/or to minimise ambient light contamination in the measurement signal. The optical filter(s) 112, 123 may be or comprise one or more of: a bandpass, multi-bandpass, long pass, short pass, or neutral density filter. In one example, a bandpass filter is placed in front of the SPAD 121 to allows only selected wavelengths (e.g. the wavelength of the light beam LB) to reach the SPAD 121.

The processing circuit 130 is in communication with the illumination system 101 and the photodetection system 120 for time of flight (ToF) data acquisition. The processing circuit 130 is configured to operate the light source 110 and control the light beam LB properties (e.g. control the operation mode, average power, and optionally repetition rate and wavelength) and control the photodetection system 120 (e.g. control the detection mode, discussed below). The processing circuit 130 is further configured to determine a ToF of photons detected over a measurement period in response to the light beam LB being directed towards the target region 20 based, at least in part, on the output of the photodetection system 120 and a timing signal from the light source 110 indicating emission of light pulses or the modulation waveform. The processing circuit 130 is then configured to process the ToF data for a given measurement period to extract one or more optical properties including the absorption coefficient μₐ and reduced scattering coefficient μₛ′, of the target region 20. One or more optical biomarker values can then be estimated based, at least in part, on the one or more optical properties, as described in more detail below.

Preferably, the processing circuit 130 is configured to repeatedly acquire ToF data over time at a rate of at least 10 Hz, to monitor the extracted optical properties and biomarker values and capture biologically relevant processes. It will be appreciated that the biomarkers monitored by the device 10 will depend on the specific use application, but by way of example, the biomarkers that can be quantified from ToF data include but are not limited to: tissue oxygen saturation, arterial oxygen saturation, venous oxygen saturation, oxy-haemoglobin, total haemoglobin, methe-haemoglobin, glycated haemoglobin, carboxy haemoglobin, deoxy-haemoglobin, lipid, water, collagen, hydration, glucose, melanin, thyrosine, thyroglobulin, cytochrome c-oxidise.

In various embodiments, the optical measurement circuit 100 is configured to perform ToF measurements at a plurality of different wavelengths to aid biomarker determination. In one example, the light source 110 is configured to output a light beam LB comprising a plurality of different wavelengths. In another example, the illumination system 101 comprises a plurality of light sources 110, each in communication with the processing circuit 130 and configured to output a light beam at a different wavelength. The wavelength of the light beam LB is variable or selectable by the processing circuit 130 controlling the output of the light source(s) 110 and/or the one or more optical filters/variable optical filters 112, 123 of the illumination system 101 and/or photodetection system 120.

The optical measurement system 100 can be configured to perform direct or indirect ToF measurements. Direct ToF methods include time-correlated single photon counting (TCSPC) and time gating using temporally uniform pulse sequences, as is known in the art. Indirect ToF methods use temporally non-uniform pulse or waveform sequences in which the output of the photodetection system 120 must be processed using an inverse transform recover the ToF data, as is known in the art. Examples of indirect ToF include spread spectrum, Fourier transform and Hadamard transform methods.

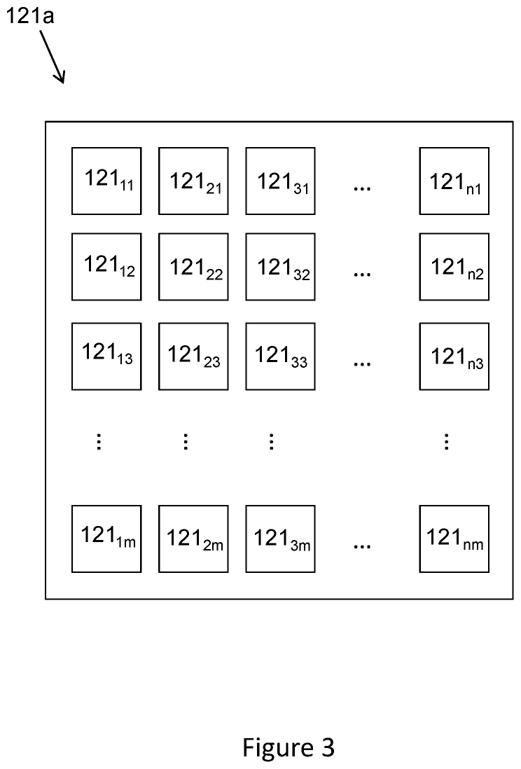

In a preferred embodiment, the optical measurement system 100 is configured to measure the ToF directly using TCSPC. The light source 110 comprises a picosecond pulsed laser (i.e. pulse width <1 ns) and the target region 20 is illuminated with a uniform pulse sequence. The photodetection system 120 comprises an n×m array of SPADs 121a, as shown schematically in FIG. 3. The timing circuitry 122 comprises a plurality of time-to-digital converters (TDCs), each TDC 122 coupled to the output of one or a group of SPADs 121nm (pixels) in the array 121a (not shown). For single photon detection, the SPADs 121s are reverse biased to operate in Geiger mode. The output of the TDCs 122 provides direct ToF data from which a histogram, or ToF distribution, of detected photons is generated by the processing circuit 130 by counting the number of photons detected at each time bin, accumulated over a plurality of light pulses. The processing circuit 130 is configured to determine a ToF of photons detected at the array 121a based with respect to a light pulse based, at least in part, on the combined output of the plurality of TDCs, and generate a ToF distribution of the sum of photons detected by the pixel SPADs 121nm of the array 121a accumulated over a measurement period.

The SPAD array 121a can be implemented by a silicon photomultiplier (SiPM), and the TDCs 122 can be operate based on capacitor charging or gated ring oscillators, as is known in the art. The array of SPADs 121a increases the count rate of the ToF measurement without pile up, as the deadtime limitation is not for the overall array 121a, but for individual SPAD 121nm in the array 121a. The count rate per SPAD 121nm is limited by the repetition rate of the light source 110, and the dead time of the respective SPAD 121nm and TDC 122. The light source 110 is operated at a repetition rate of at least 10 MHz to avoid pile up in the measured ToF data from each pixel SPAD 121nm. Thus, each pixel SPAD 121nm has independent statistics and when their outputs summed up, they produce a high count rate without pile up.

As with traditional direct ToF techniques, in one implementation the pulsed laser 110 is operated at a repetition rate fᵣₑₚ that is high enough to avoid pile up, but low enough to avoid wrapping of the ToF signal. In practice, this typically means fᵣₑₚ is between 10 MHz and 100 MHz. The count rate per pixel 121 nm is then approximately 1% of fᵣₑₚ, i.e. between 10⁵ and 10⁶ cps. As such, count rates of over 1 Gcps without pile up can be achieved using a 100 MHz repetition rate and over 1000 pixels. To reduce the data output, ToF data from the array 121a can be processed (e.g. histogramming and summed/averaging) at wafer level.

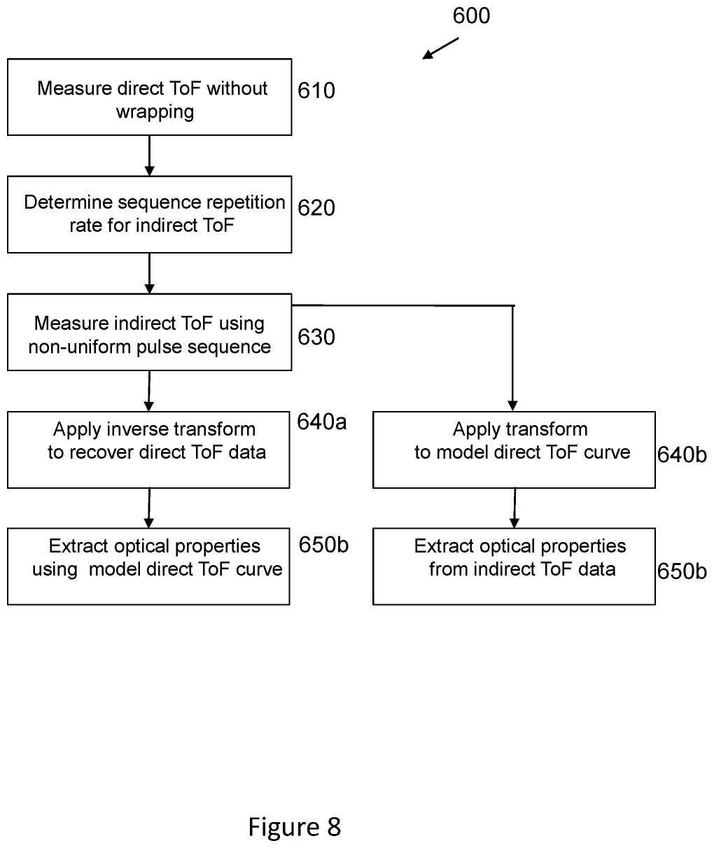

In other embodiments, the optical measurement system 100 can be configured to measure ToF directly using time gating, in which TDCs 122 are not required, or indirectly using temporally non-uniform pulse sequences (as described in more detail with reference to FIG. 8)

The processing circuit 130 is configured to extract optical properties of the target region 20 from analysis of the shape of the ToF distribution. The temporal profile of laser pulses travelling through biological tissue is strongly shaped and broadened by the tissue optical properties. Specifically, light injected into the target region 20 undergoes absorption and scattering, which randomises the photon paths within the tissue, such that photons detected at a given distance d from the injection point P1 will have sampled a range of different depths and path lengths through the tissue, indicated schematically by the region labelled D in FIG. 2. As a result, the ToF distribution of photons re-emitted at a given distance from the injection point P1 is temporally broadened compared to the instrument response function (IRF) because of scattering and is attenuated with an exponentially decreasing tail due to absorption. The measured ToF distribution is a convolution of the IRF and the impulse response of the target region 20. This is illustrated in FIG. 4 which shows an example of a measured ToF distribution (curve A) and the IRF (curve C). Absorption affects the slope of the tail of the ToF distribution, whereas scattering temporally shifts and broadens the peak. In this way, ToF measurements naturally decouples the effects of absorption and scattering, and the optical properties of the tissue, i.e. absorption coefficient μₐ and reduced scattering coefficient μₛ′, can be extracted from the shape of the measured ToF distribution.

ToF data can be analysed in various ways to extract optical properties (absorption, scattering). The most common way is to fit the measured ToF distribution with a model or predicted ToF curve derived from a theoretical model of light propagation in diffusive media considering the source-detector geometry, R(t, μₐ, μₛ′), convolved with the instrument response function (IRF), according to:

$${ToF}_{theoretical} ( t ) = IRF ( t ) * R ( t , {μ}_{a} , {μ}_{s}^{′} ) \qquad ( 1 )$$

where * denotes a mathematical convolution. The theoretical model, R(t, μₐ, μₛ′), represents the impulse response of the medium can be defined by an analytical formula or Monte Carlo techniques. The theoretical model can take into account a single layer of diffusive media or multiple layers e.g. a bulk layer 20b and superficial layer 20s.

FIG. 4 shows an example of a measured ToF distribution (curve A) and a fitted model ToF curve (curve B) obtained from an analytical solution to the diffusion equation convolved with the IRF (curve C). By adjusting the variables of the model to minimise the residuals, the optical properties μₐ and μₛ′ can be readily extracted.

From the extracted optical properties and applying the Lambert Beer Law together with the known extinction coefficients of the tissue constituents/biomarkers, the concentration of key tissue constituents can be determined. It is also possible to use machine learning to extract optical properties and/or optical biomarkers from the shape of the ToF distribution.

If the extinction coefficient ε of each biomarker/tissue constituent is known at the wavelength of interest, the biomarkers or tissue constituents can be quantified by fitting a linear combination of them, according to:

$${μ}_{a} ( λ ) = {\sum }_{i} {c}_{i} {ε}_{i} ( λ ) \qquad ( 2 )$$

where εᵢ is the extinction coefficient and cᵢ is the concentration of the tissue constituent.

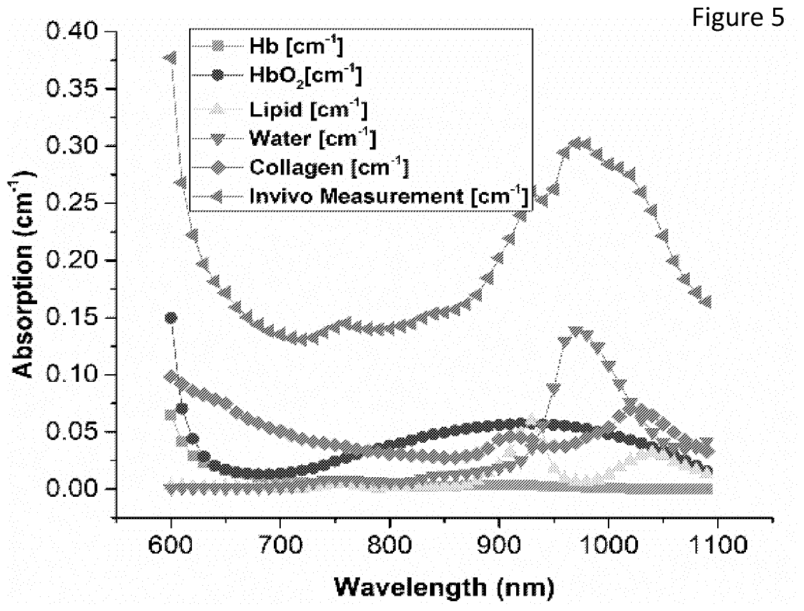

FIG. 5 shows an example absorption spectrum of a human tissue sample obtained from in vivo TD DOS measurements obtained at a multiple different wavelengths (upper most data), and the resulting absorption spectra of various biomarkers (oxy, deoxy-haemoglobin, lipids, water and collagen) extracted using equation 2.

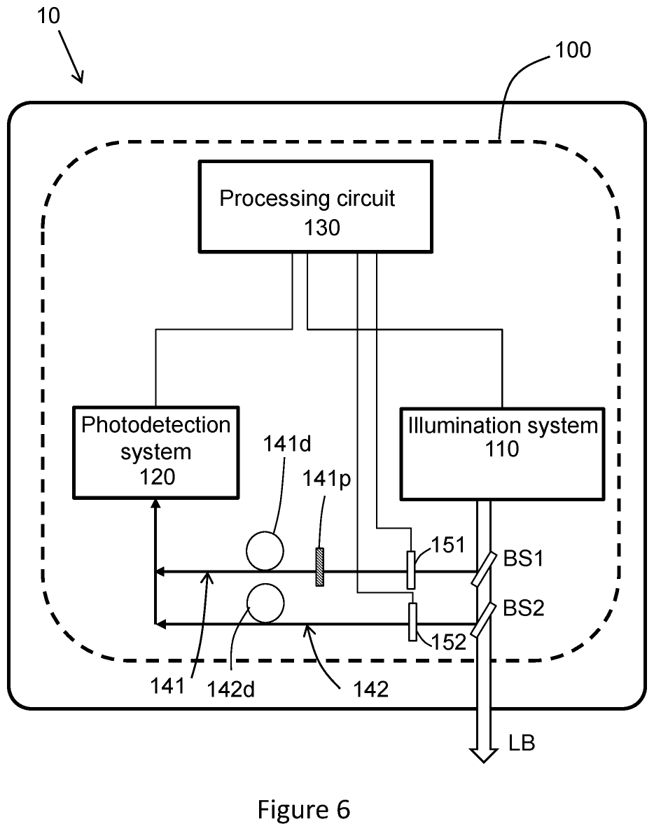

FIG. 6 shows an embodiment of the wearable device 10 configured to perform standardised time domain diffuse optical spectroscopy according to an embodiment of the invention, in which the optical measurement system 100 further comprises a first reference optical path 141 and a second reference optical path 142 between the illumination system 101 and the photodetection system 120 for coupling at least a portion of the light beam to the photodetection system 120 without interacting with the target region 20, to provide respective first and second reference signals. The first reference optical path 141 is used to measured ToF data from of a tissue mimicking phantom 141p with known optical properties (μₐ, μₛ′) to calibrate the device 10 and monitor performance over time. The second reference optical path 142 is used to measure the IRF for use in extracting the optical properties of the target region 20 and on-chip monitoring of drift and distortion of the source/detector outputs. On chip calibration/correction for drift of the light source/detector outputs is especially important in wearable devices, where the source detector separation may be small, e.g. <1 cm.

TD DOS requires knowledge of the IRF to produce a model ToF curve and extract optical properties from the ToF data (e.g., see equation 1). In more cumbersome laboratory TD DOS systems, acquiring the IRF is quite straightforward, and can be achieved simply by coupling the source and detector fibers or using a parallel arm that diverts part of the beam directly to the detector, so that the signal does not travel inside the target region. In such cases, where the optical set up is stable, the IRF acquisition is typically not performed at the same time as the ToF of photons from the target region, but is performed before or after the tissue measurement. However, IRF acquisition in a wearable device which is typically small is not straightforward, and may be needed more regularly than in a lab set up as a result of device motion and other environmental factors.

Typically, phantoms are relatively large in size compared to commercial wearable devices (e.g. several cm in diameter). A micro-phantom suitable for incorporating into the first reference optical path 141 of the wearable device 10 can be produced using highly scattering material (to get more scattering from small area) to create required temporal dispersion in ToF distribution similar to human tissue.

The first and second reference optical paths 141, 142 may be implemented using one or more optical waveguides, fibres or light pipes for guiding the light pulses to the photodetection system 120. In practice, only a small fraction of the light beam is coupled to the photodetection system 120 to avoid saturating the SPADs 121nm. Further, the reference signals may not be required all the time. As such, the optical measurement system 100 include one or more optical elements/filters to control and/or attenuate the light reaching the photodetection system 120 along the first and second reference optical paths 141, 142 (e.g. optical filters 112, 123).

In the embodiment shown in FIG. 6, beam splitters BS1, BS2 are used to direct a portion of the light beam LB along the first and second reference optical paths 141, 142. In this case, it is possible for light reach the photodetection system 120 via both the target region 20 and the reference optical paths 141, 142, which may cause saturation of the detectors 121 and/or unwanted signal distortion if the signals overlap in time. Accordingly, the optical measurement system 100 comprises first and second optical elements 151, 152 configured to selectively allow/prevent (i.e. shutter) the portion of the beam to reach the photodetection system 120 along the respective first and second reference optical paths 141, 142. In this way, the optical measurement system 100 can be switched between a measurement mode, in which the ToF of photons from the target regions 20 is measured, a first reference mode in which the ToF of photons from the phantom 141p is measured, and a first reference mode in which the IRF is measured.

In operation, the processing circuit 130 is configured to send a control signal to the first and second optical elements 151, 152 to change a state of the first and second optical elements 151, 152 and selectively couple a portion of the beam to the photodetector 120. In a preferred embodiment, the first and second optical elements 151, 152 comprise an electro-absorption modulator (EAM). However, it will be appreciated that the first and second optical elements 151, 152 can be any element configured to selectively direct, transmit or block the portion of the beam along the respective reference optical path 141, 142, including but not limited to: a moveable mirror, a movable beam splitter, a moveable attenuator, adjustable attenuator, a shutter, a liquid crystal device, or an electro-absorption modulator.

In the measurement mode, both optical elements 151, 152 are configured to block light from reaching the photodetection system 120 along the reference optical paths 141, 142.

In the first reference mode, the processing circuit 130 is configured to control the first optical element 151 to transmit light and second optical element 151 to block light, extract one or more optical properties of the phantom 141p from the shape of the measured ToF distribution using a similar approach as described above, and determine, based on a comparison of the extracted one or more optical properties with the known optical properties of the phantom 142p, a first calibration factor to apply to the optical properties determined from the ToF measurements of target region 20. The first calibration factor is stored by the processing circuit 130.

In the second reference mode, the processing circuit 130 is configured to control the first optical element 151 to block light and second optical element 151 to transmit light, and measure the IRF. The processing circuit 130 stores the measured IRF for use in extracting the one or more optical properties of the target region 20 and phantom 141p, and/or for compensating for drift/distortion in the light source output/detector efficiency.

Specifically, the processing circuit 130 uses the stored IRF to generate the model ToF curve that is fit to the measured ToF distributions, and the processing circuit 130 can also determine, based on the stored IRF, a second calibration factor to correct for drift in the optical measurement system (e.g. drift in the light source power output, detector efficiency, other drifts etc.). The second calibration factor is also used to determine an overall attenuation of light by the target region 20 (described further below).

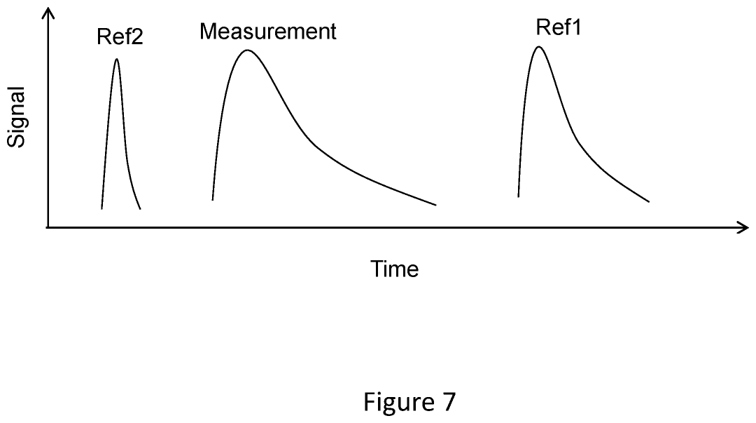

A portion of the light beam LB can still directed/transmitted to the target region 20 in the first and second reference modes, potentially causing unwanted temporal overlap of the reference and measurement signals. Various active and/or passive solutions can be implemented in the device 10. In one active approach, a third optical element (not shown) can be arranged downstream of the reference optical paths 141, 142, configured to selectively block light from reaching the target region 20 in the first and second reference modes. FIG. 6 shows a passive solution whereby the first and second reference optical paths 141, 142 include respective delay lines 141d, 142d configured to temporally separate the first and second reference signals (Ref1, Ref2) from the measurement signal from the target region 20, as shown schematically in FIG. 7. This allows simultaneous acquisition of the reference signals and IRF/phantom ToF data and the ToF data from the target region 20 in the first and second reference modes. A passive solution also reduces the power consumption compared to an active approach.

In practice, the delay lines 151, 152 can be implemented by a loop or length of optical fibre or other flexible light guide, e.g. incorporated into the body or wrist band of the device 10 (in the case of a smart watch).

In another passive approach (not shown), if the measurement and second reference signals overlap in the second reference mode, the IRF can be extracted from the second reference signal by comparison to the measurement signal in the measurement mode (which contains no reference signals). Because the first reference signal associated with the IRF is typically of much larger in amplitude (orders of magnitude) than the measurement signal (because it is not attenuated by the target region 20), it can be extracted by signal processing.

In an alternative embodiment (not shown), the second reference optical path 142 can be omitted, and the first reference optical path 141 can be used to extract the IRF and the optical properties of the phantom 141p and determine the first and second calibration factors. In this case, the IRF is extracted from the first reference signal using the known optical properties of the phantom 142. For example, the impulse response of the phantom 141p is known or can be calculated using its known optical properties (μₐ and μ′ₛ), and the IRF can be extracted either by deconvolving the phantom impulse response from first reference signal, by comparing it to a database of model ToF curves generated from the phantom impulse response and different IRFs, or by simulating different IRFs. The optical properties of the phantom 141p can then be extracted from first reference signal using the measured/extracted IRF.

Measurements performed on the phantom 141p in the first reference mode can be performed regularly, e.g. periodically or in response to a user command/input, and serve the purpose of regular calibration and performance assessment of the wearable device 10, and/or life-time surveillance to meet medical device regulatory (MDR) requirements. In an embodiment, the processing circuit 130 is configured to periodically make the measurement on the internal microphantom 141p and do self-calibration or referencing. In an alternative implementation, the first reference optical path 141 can be incorporated in a charging dock of wearable device 10, to enable regular device calibration while charging (not shown).

Use of a micro-phantom reference optical path 142 is not only limited to TD based wearable devices, but can also be used along with other DOS techniques such as CW and indirect ToF methods such as frequency domain (FD) methods for the purpose of regular performance assessment/calibration of wearable device or life-time surveillance to meet MDR requirements.

In theory, FD data is Fourier's transform of ToF data. In FD techniques, modulated CW lasers are used instead of pulsed lasers to deliver a sequence of sinusoidal waveforms of differing frequencies. A phantom for calibrating the system and fitting the optical properties of measurement on tissue is still required but has not been demonstrated in a wearable device scale. As such, the micro-phantom/reference architecture that is inbuilt in the wearable device 10 to perform this operation as shown in FIG. 6 can be applied to FD based devices.

Similarly, in CW based devices the reference/micro-phantom optical path 141 can be used to correct for the drift in the light source and the detector efficiency, and as a reference measurement to increase the accuracy of the CW system. Regular reference phantom measurements can be used to correct errors in measured biomarkers and improve accuracy using the first and second calibration factors.

As described above, the use of a SPAD array 121a enables high count rate ToF data to be acquired without pile up. In traditional direct ToF measurements such as TCSPC and time gating, the laser repetition rate is typically limited to 100 MHz to avoid wrapping of the ToF data, which limits the photon count rate per pixel to about 10⁵ to 10⁶ counts per second.

In an embodiment, the SPAD array 121a is segmented, or divided up into a plurality of (spatial) segments, each segment comprising a sub-group of pixel SPADs 121nm of the array 121a that is configured to output/provide a separate signal for processing (not shown). In this embodiment, the optical measurement system is configured to couple scattered light exiting the target region to a first segment of the array 121a to provide the measurement signal, to couple light propagating along the first reference optical path 141 to a second segment of the SPAD array 121a to provide the first reference signal, and to couple light propagating along the second reference optical path 142 to a third segment of the array 121a to provide the second reference signal. In this way, the optical measurement system can measure the measurement signal and first and second reference signals simultaneously using different segments of the SPAD array 121a. In this case, rather than coupling together the outputs of each TDC 122, the outputs of the TDC(s) 122 associated with each segment are coupled together to provide the separate ToF signals. Separate TOF distributions can then be generated for each ToF signal.

FIG. 8 shows a method 600 of performing standardised indirect ToF measurements at high repetition rate according to an embodiment. The method 600 involves the use of compression techniques, such as the spread-spectrum method, in combination with a high count rate photodetection system 120 to reduce the effect of noise described above and increase the dynamic range of the ToF data. As compared to prior art indirect ToF measurements using conventional photodetection systems, e.g. spread spectrum (see K. I. Papadimitriou et al., “A spread spectrum approach to time domain near infrared diffuse optical spectroscopies”, Physical Measurements, 35, 1469 (2014)), which have a dynamic range of the order of a factor 10, the proposed method 600 increases the dynamic range to around 10⁴-10⁵ through use of the high count rate photodetection system 120. Indirect ToF may be advantageous over direct ToF in certain cases. In particular, it allows ToF measurements to be effectively implemented by using cheaper light sources like modulated CW instead of gain switched/mode locked pulsed lasers used in direct ToF. In addition, the high average power due to the high duty cycle of spread spectrum sequence will increase the overall signal count which provides high SNR for software-based CW measurements (e.g. for fast PPG signal measurement) described in more detail below. The average power can also be controlled by tailoring the pulse/waveform sequence used.

In this embodiment, the light source 110 again has a variable/dynamic repetition rate in the range 10 MHz-10 GHz, and the photodetector system 120 comprises a SPAD array 121a with TDCs 122, as described above. The light source 110 is a high-speed telecommunications modulated CW laser or LED operating at the wavelength of interest. Modulation can be achieved at the source level or it can be done externally by using a modulator, as is known the art. For example, the light source 110 may be or comprise a low cost commercially available optical transceiver module configured for operating at data rates of up to 10 Gbits/second.

At step 630, the processing circuit 130 is configured to control the light source 110 to operate at a dynamic repetition rate according to a predefined temporally non-uniform pulse sequence, and measure an indirect ToF distribution of photons detected from the target region 20 based on the output of the SPAD array 121a. Part of the light can be used for IRF/phantom measurements as described above. In an embodiment, the spread spectrum technique is implemented, and the temporally non-uniform pulse sequence is a pseudorandom binary sequence (PRBS) that repeats at a certain sequence repeat rate fₛᵣₑₚ. The sequence repeat rate fₛᵣₑₚ determines the width of the ToF window and is therefore equivalent to the pulse repetition rate fᵣₑₚ in conventional direct ToF measurements. At least some pulse periods in the PRBS pulse sequence are shorter than the temporal width of the ToF distribution of photons detected from the target region 20, such that the measured indirect ToF data is substantially wrapped. In other embodiments, the non-uniform pulse sequence can be a wavelet transform sequence, Hadamard pulse sequence, or Fourier sinusoidal sequence.

The processing circuit 130 generates the indirect ToF distribution in the same way as in the direct ToF approach, however, the indirect ToF data does not resemble the conventional ToF distribution as shown in FIG. 4 with a well-defined rise and fall. In the case of spread spectrum, the indirect ToF data is the convolution of the transmitted PRBS with the IRF and the impulse response of the target region 20. The conventional direct ToF distribution can be recovered by applying the appropriate inverse transform for the compression method used.

At step 640a, the processing circuit 130 is configured to transform the measured indirect ToF distribution by applying an inverse transform. In the spread spectrum method, step 640a comprises cross-correlating the indirect ToF with the PRBS to deconvolve it. This transforms the indirect ToF data to direct ToF data that resembles FIG. 4 (if the sequence length is longer than the width of the ToF distribution). At step 650a, the processing circuit 130 is configured to extract the one or more optical properties of the target region 20 from the shape of the recovered direct ToF distribution by fitting with a model ToF curve, i.e. in direct ToF space as described above.

Alternatively, the processing circuit 130 can extract the one or more optical properties of the target region 20 directly from the shape of the indirect ToF distribution, i.e. in indirect ToF space. This may provide more accurate results, as applying an inverse transform to the indirect ToF data, in certain cases, can introduce noise. In this case, at step 640b, the processing circuit 130 is configured to apply a transform to the model ToF curve based on the properties of the nonuniform pulse/waveform sequence to generate a modulated/indirect model ToF curve that can be compared to the measured indirect ToF data. For example, in the spread spectrum method, step 640b comprises convolving the model ToF curve with the PRBS, according to:

$${ToF}_{modtheoretical} ( t ) = IRF ( t ) * PRBS * R ( t , {μ}_{a} , {μ}_{s}^{′} ) \qquad ( 4 )$$

$$or$$

$${ToF}_{modtheoretical} ( t ) = {IRF}_{PRBS} ( t ) * R ( t , {μ}_{a} , {μ}_{s}^{′} ) \qquad ( 5 )$$

where IRF_{PRBS}(t) is the IRF function measured with a PSBS according to the spread spectrum method (which is effectively the IRF convolved with the PRBS), e.g. using the first or second reference optical path 141, 142.

At step 650b, the processing circuit 130 then extracts the one or more optical properties of the target region 20 from the shape of the measured indirect ToF distribution by fitting with the modulated/indirect model ToF curve.

In one implementation, the length of the PRBS (1/fₛᵣₑₚ) is greater than the width of the ToF distribution to avoid wrapping in the recovered direct ToF data, as is conventional in the art.

In addition to using one or more filters 112 or controlling the power output of the laser 110, the average power on the target region 20 can be controlled by introducing the delay between each repetition of the PRBS sequence. A delay the same in length as the PRBS sequence will reduce the power by half compared to no delay. In another embodiment, the power is controlled by changing the mid-point of binary state of PRBS sequence.

In another embodiment, the method 600 is implemented using a Fourier transform method. In this case, at step 630, the processing circuit 130 is configured to control the light source 110 to output a sequence of sinusoidal waveforms at set of selected frequencies (40-300 MHZ). The photodetection system 120 is the same as described above (a high count rate SiPM 121a with parallel TDCs 122, or high speed ADCs connected to the SiPM). The processing circuit 130 contains a digital homodyne detection system (lock in detection) to extract the amplitude and phase of the detected signal. The device 10 includes first and second reference optical paths 141, 142 for calibrating the overall attenuation of photons by the target region 20. In particular, to enable accurate estimation of tissue optical properties, the phase and amplitude of the detected signal needs to calibrated with the reference object, which can be achieved using the measurements on the phantom 141p in the first reference mode. The optical properties of target region 20 can be extracted in the indirect ToF space or direct ToF space. In the former case, by fitting the obtained three parameters (amplitude, phase, attenuation) at multiple frequencies fitted to a model derived from a solution to the diffusion equation in the frequency domain, as is known the art. In the latter case, the information of phase and amplitude can be Fourier transformed into direct ToF space to recover the direct ToF distribution and the fitting procedure is carried out using the model ToF curve in direct ToF space, as described previously.

The indirect ToF measurements in method 600 can be performed at multiple source-detector distances to enable multi-layer optical properties assessment of target region 20 (not shown).

Method 600 in combination with the SPAD array 121a, allows the count rate of ToF measurements to be increased further without pile up by use of high repetition rate lasers. This increases the dynamic range of the measurement while also maintaining high temporal resolution as each pixel 121 nm of the array has a small area, preventing long diffusion paths in the detector diffusion region. High dynamic range is particularly important for measuring small changes in physiological or pathological conditions with increased penetration depth. Increased count rate and dynamic range also means the device 10 is less sensitive to ambient light reaching the photodetector system 120.

In another embodiment of the device 10, the optical measurement system 100 is configured to perform (direct or indirect) ToF measurements as well as continuous wave (CW) measurements. This provides a hybrid ToF/CW device that can leverage the advantages of both technologies. For example, CW measurements can be used to monitor small relative changes in optical properties such as PPG or other highly varying physiological signal by virtue of the high signal to noise ratio of the CW measurement, and ToF measurements can be used to complement the CW technology by providing calibration factors, error correction and/or motion correction, as described in more detail below.

In an embodiment, the processing circuit 130 is configured to implement a software CW mode, in which CW data/information is extracted from the measured (direct or indirect) ToF distribution of detected photons. CW data is extracted from the integrated (total) photon count of the ToF distribution which is equal to the amount of scattered light detected in the measurement period and represents CW information. In this embodiment, the processing circuit 130 can acquire both ToF data and CW data at each measurement period in real time. Software CW mode can be implemented when using modulated or pulsed light sources 110, and with direct or indirect ToF measurements. The processing circuit 130 is configured to process ToF mode data in software CW mode to extract fast varying signals, e.g. PPG or hemodynamic changes with high SNR.

In one implementation, the device 10 is configured to perform spread spectrum indirect ToF measurements in ToF mode according to method 600 described above and implement a software CW mode to extract CW data from the measured indirect ToF data (sum of all spread spectrum signal). In the software CW measurement mode, the processing circuit 130 is configured to measure a series of indirect ToF distributions in response to the PRBS being directed to the target region 20, extract a timecourse of CW data (the amount of scattered light detected from at each measurement period) from the series of indirect ToF distributions. The processing circuit 130 is then configured to determine one or more optical biomarker values based, at least in part, on one or more optical properties of the target region 20 extracted from the measured CW data timecourse, as is known the art.

A software CW mode may be particularly advantageous with indirect ToF measurements, such as spread spectrum, which can be implemented using modulated CW light sources which are cheaper, smaller and consume less power than pulsed light sources used in direct ToF. In particular, by using a high duty cycle PRBS high average powers can be achieved comparable to traditional CW measurements, making the software CW mode suitable for high count rate CW measurements of fast varying physiological signals e.g. for monitoring PPG signals in a CW oximeter mode. At the same time, the measured indirect ToF distribution can be used to derive calibration and corrections to compensate for scattering, superficial layer and motion artefacts in the CW data (see below).

In another embodiment, the device 10 is configured to include a hardware CW mode. A hardware CW mode can be implemented in a number of ways. In one implementation, the same light source 110 and photodetector 121 can be used to perform both ToF and CW measurements. These are referred to as a hybrid light source 110′ and photodetector 121′. In this case, the processing circuit 130 is configured to operate the hybrid light source 110′ and the photodetector 121′ in ToF mode and CW mode. In the TOF mode, the light source 110′ outputs a beam of light pulses and the photodetector 121′ operates in a single photon detection mode for measuring a ToF of photons detected in response to a light pulse. In the CW mode, the light source 110 is configured to output a continuous beam of light, and the photodetector 121 is operated in conventional detection mode for measuring an amount of scattered light in response to the continuous beam of light. Pulsed laser sources can typically be run in CW mode. For example, instead using gain switching technique to pulse a laser diode, it can simply be biased above the laser threshold to output CW light. Likewise, a SPAD 121 can be operated in “Geiger mode” for low count rate single photon detection and conventional avalanche photodiode mode or photovoltaic mode for CW measurements by changing the bias on the detector 121.

In another implementation, the optical measurement system 100 can have separate dedicated light sources and/or photodetectors for performing ToF and CW measurements. For example, in addition to a SPAD 121 or SPAD array 121a for ToF measurements, the photodetection system 120 can comprise a second (CW) photodetector 125 to detect scattered light from the interaction of a continuous beam of light with the target region 20 for CW measurements. The second photodetector 125 can be a conventional photodiode or avalanche photodiode, SPAD or SPAD array (e.g. a SiPM). The CW light can be produced from the hybrid light source 110′ operated in CW mode, or a second (CW) light source 115 configured to illuminate the target region 20 with a continuous beam of light, such as a CW laser or LED. The second photodetector 125 and/or light source 115 are in communication with and controlled by the processing circuit 130.

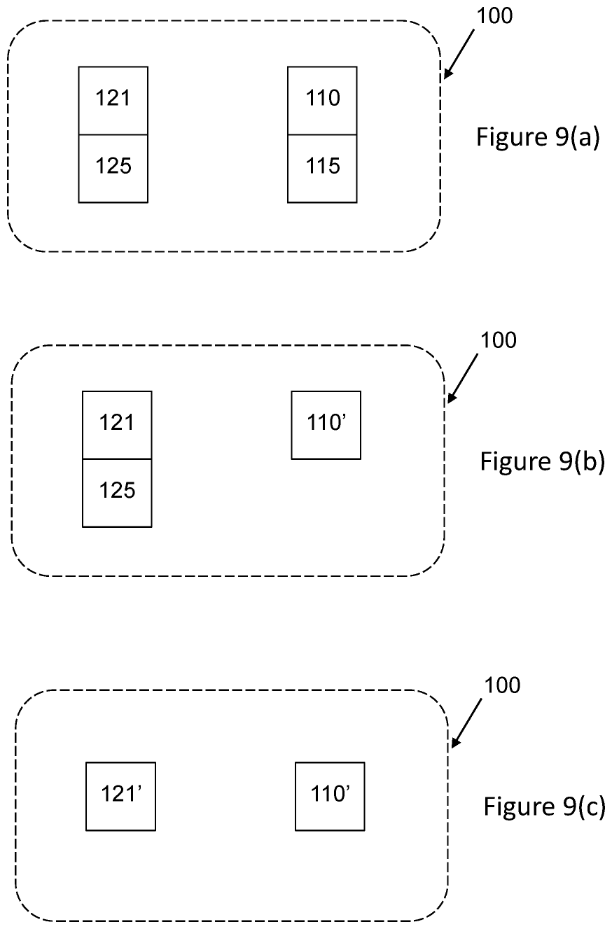

Various configurations for a hybrid CW/ToF device 10 are possible. FIG. 9(a) shows an embodiment where the optical measurement system 100 comprises a first light source 110 and first photodetector 121 for ToF measurements, a second light source 115 and second photodetector 125 for CW measurements. The first and second light sources 110, 115 and the first and second photodetectors 121, 125 are configured in pairs. Preferably, the first and second light source 110, 115 are configured to illuminate the target region 20 at substantially the same injection point P1, and the first and second photodetectors 121, 125 are configured to detect light from the same detection point P2. For example, light from the pair of light sources 110, 115 can be routed to substantially the same injection point P1 using optical fibres. Similarly, light can be collected and routed to the pair of photodetectors 121, 125 using optical fibres. Preferably, the pairs of light sources 110, 115 and photodetectors 121, 125 are substantially co-located.

FIG. 9(b) shows an embodiment where the optical measurement system 100 comprises a hybrid light source 110′ operable in both ToF and CW modes, and a pair of dedicated first and second photodetectors 121, 125 for ToF and CW measurements.

FIG. 9(c) shows an embodiment where the optical measurement system 100 comprises a hybrid light source 110′ and photodetector 121′ operable in both ToF and CW modes.

Having a range of source-detector positions and separations d provides different depth sensitivity to the ToF data, and probes different areas of the target region 20 providing spatial sensitivity e.g. segmentation of extracted optical properties of the target region 20. The latter can provide segmentation of superficial layer 20s properties that in turn improves motion artefact correction (see below). It also enables accurate quantification of multilayer optical properties (i.e. bulk 20s and superficial layer 20s optical properties) by exploiting a combination of CW and ToF models at range of source detector separations. Specifically, the shape of the ToF distribution is most sensitive to superficial layer 20s scattering properties and bulk layer 20b absorption properties. Meanwhile, the overall attenuation form the target region 20 is dependent primarily on the superficial layer 20s absorption properties and a multi-distance CW measurements carries information of bulk layer 20b scattering properties. As such, the combination of ToF, overall attenuation and multi-distance CW measurements will enable accurate estimation of the superficial 20s and bulk layer 20b optical properties by exploiting sensitivity of these three measurements to the different layers. For this a combination, multi-distance two-layer model with inputs of overall attenuation, ToF distribution at multi-distance is used.

The processing circuit 130 is configured to dynamically switch the optical measurement system 100 between a ToF measurement mode and a hardware CW measurement mode to perform ToF and CW measurements in real time that can be compared. In the hardware CW measurement mode, the processing circuit 130 is configured to operate the appropriate light source 110′, 115 to illuminate the target region 20 with a continuous beam of light and measure a timecourse of the amount of scattered light detected (by the appropriate photodetector 121′, 125) from the target region 20 in response to the continuous beam of light (CW data). The processing circuit 130 is then configured to extract determine one or more optical biomarker values based, at least in part, on one or more optical properties of the target region extracted from the measured timecourse, as is known the art.

The processing circuit 130 is configured to apply one or more correction factors to the one or more optical biomarker values determined in the hardware or software CW measurement mode, or process the CW data, based, at least in part, on one or more static and/or dynamic optical properties or optical biomarker values determined in the ToF measurement mode. This may improve any CW based biomarker assessment.

In one example, a correction factor is derived from the reduced scattering coefficient μₛ′ of the target region 20 measured in ToF mode. This can be used to compensate for or eliminate the scattering contribution to optical properties estimated from CW measurements (where μₐ and μₛ′ inherently coupled), e.g. as this varies among different subjects.

In another example, a correction factor is derived from the absorption coefficient μₐ of a superficial layer 20s of the target region 20 determined in ToF mode, described in more detail below. This can be used to compensate for superficial layer distortion to optical properties/biomarker values estimated in CW mode.

In another example, the processing circuit 130 is configured to process the CW data to remove motion artefacts detected in ToF mode, as described in more detail below.

In various embodiments, the wearable device 10 is configured to detect the presence of and determine one or more optical properties of a superficial layer 20s of the target region 20. A superficial layer 20s may be any layer in or on the tissue that the incident light beam LB must pass through before reaching the bulk of tissue 20b, as shown in FIG. 2. For example, the superficial layer 20s may be an outer layer of the tissue, such as melanocytes in the epidermis that provide skin pigmentation, or a nail or nail polish.

The main effect of a superficial layer 20s is to attenuate light which reduces the amount of scattered light reaching the photodetector. CW measurements are based on the amount of scattered light reaching the detector (i.e. signal amplitude) and so are particularly sensitive to the presence and properties of a superficial layer 20s. Indeed, skin pigment related distortion is one of the largest sources of error in CW-based measurements of biomarker values, e.g. dark skin attenuates more than light skin which biases the results. By contrast, ToF measurements of optical properties are inherently less sensitive to the superficial layer 20s properties because the optical properties are extracted from the shape of a ToF curve, not a signal amplitude. The shape of the ToF curve is measurements are largely unaffected by the presence of a superficial layer 20s. However, optical properties of the superficial layer 20s can still be quantified from analysis of the overall attenuation, and in some cases the shape of the ToF curve. The extracted superficial layer properties (absorption and/or scattering) can be static or dynamic and can be used as an additional biomarker to improve the analysis of the target region 20 and/or indicator of motion. Further, as referenced above, the superficial layer properties can be used to compensate for associated errors in CW measurements, which is described in more detail below.

The primary effect of a superficial layer 20s on ToF data is to change the overall attenuation of the ToF curve without changing the shape, i.e. the amplitude of the ToF curve. This is particularly true at relatively large source-detector separations d, e.g. d>2 cm, and relatively thin superficial layers 20s. The secondary effect, which can manifest at short source detector separations d (e.g. d<1 cm) and/or when the superficial layer 20s is sufficiently thick, is to contribute to scattering and cause additional attenuation of early photons in the ToF curve, which changes the shape of the ToF curve.

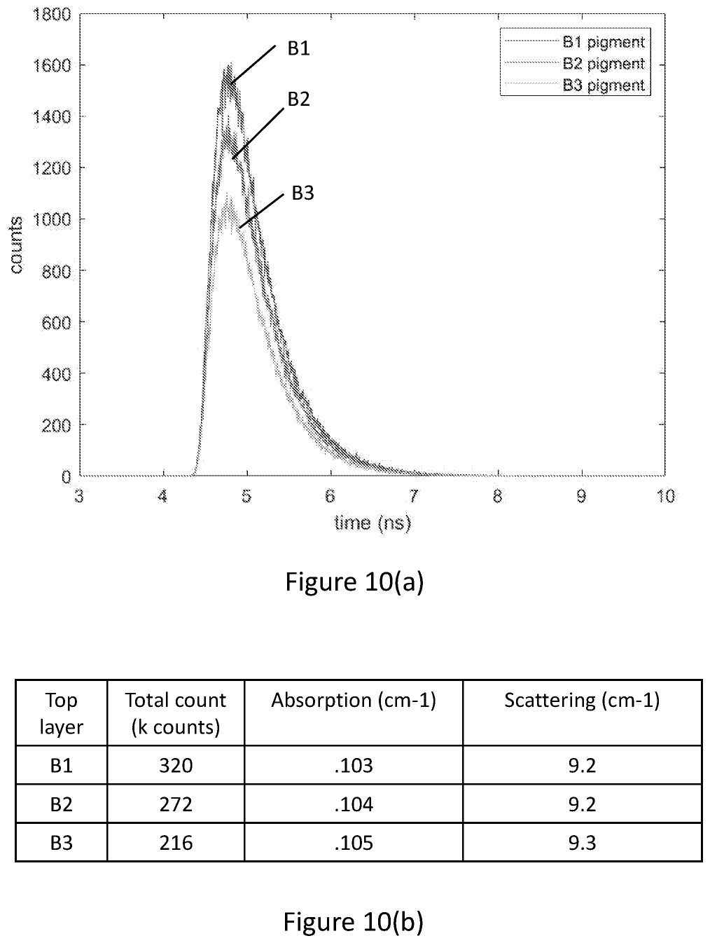

To demonstrate the primary effect, FIG. 10(a) shows experimental ToF distributions obtained from direct ToF measurements of a two-layer tissue mimicking phantom with a 5 cm thick bottom layer 20b and a 1 mm thick superficial layer 20s of varying absorption properties: curve B1, μₐ=0.05 cm⁻¹; curve B2, μₐ=0.2 cm⁻¹; curve B3, μₐ=0.7 cm⁻¹. The scattering properties of the superficial layer is the same in each case (μₛ′=10 cm⁻¹). The source-detector separation was d=2 cm. FIG. 10(b) shows the optical properties μₐ and μₛ′ extracted from the shape of the ToF curves from a fit to an analytical solution to the diffusion equation convolved with the IRF (as described above). As expected, the changing absorption properties of the superficial layer 20s have minimal effect on the optical properties estimated by ToF measurements, however, the overall signal (total counts) was attenuated by more than 33% as the absorption coefficient of the superficial layer 20s increases.

These phenomena can be used to predict one or more optical properties of the superficial layer 20s. Moreover, where the device 10 is configured to perform both ToF measurements and CW measurements (with a hardware or software CW mode, see above), the superficial layer 20s properties extracted from ToF measurements can be used to compensate for distortions in CW measurements.

Analysis of the signal attenuation can be used to detect the presence and/or determine the attenuation (absorption) caused by the superficial layer 20s. In this embodiment, the device 10 includes the first and/or second reference optical paths 141, 142 to measure/extract the IRF and calibrate the ToF data for drift in the light source 110 output and detector efficiency using the second calibration factor. The overall signal attenuation is determined by comparing the total photon count of the calibrated ToF distribution (CW information) and the output of the light source (e.g. the average power P, and wavelength λ. For example, the total photon count of the ToF distribution divided by the measurement period can be compared to the average power divided by the photon energy (hc/λ, where c is the speed of light). The overall signal attenuation can also be determined by comparing the total photon count of the measured ToF distribution and the total photon count of the measured IRF. Alternatively, in embodiments where the device 10 has a hardware CW mode, the CW data obtained in hardware CW mode can be used to determine the overall attenuation of photons.

In an embodiment, the processing circuit 130 is configured to determine one or more optical properties of the superficial layer 20s by comparing the overall attenuation of photons by the target region 20 with an effective attenuation. The effective attenuation of photons by the target region 20 can be determined from the geometric mean of the absorption and reduced scattering coefficients μₐ and μₛ′ extracted from the shape of the measured ToF distribution through fits with a model ToF curve derived from a theoretical single or multi-layer diffusive media model convolved with the IRF as described above. Effective attenuation can be given by a modified beer lamberts law:

$$I = {I}_{0} {e}^{{μ}_{a} * DPF * d + G} , \qquad ( 6 )$$

where d is source-detector distance, DPF and G are dependent on the system parameters and geometry. The DPF for a semi-infinite (reflectance geometry) model is given by

$${DPF}_{semi infinite} = \frac{\sqrt{3 {μ}_{s}^{′}}}{2 \sqrt{{μ}_{a}}} \frac{( d \sqrt{3 {μ}_{a} {μ}_{s}^{′} )}}{( d \sqrt{3 {μ}_{a} {μ}_{s}^{′} + 1} )} , \qquad ( 7 )$$

for a source-detector separation of unit length of diffuse media, or it can be calculated by Monte Carlo techniques by considering system parameters and geometry of target region 20. To compare to the overall attenuation, the effective attenuation as described above can be used or it can be estimated by considering absorption with an average path length in the target region 20 determined from the average ToF in the measured ToF curve.

If the overall attenuation is greater than expected based on the effective attenuation, this will indicate the presence of a superficial layer 20s. Further, the attenuation caused by the superficial layer 20s can be quantified by comparison of the overall attenuation with the effective attenuation. Specifically, fitting with a model ToF curve provides a scattering properties μₛ′ in the superficial and bulk layer 20b layer 20s, as well as absorption μₐ in the bulk layer 20b, while the overall attenuation is due to absorption and scattering in both the superficial layer 20s and the bulk layer 20b. Absorption in the superficial layer 20s affects only the overall counts in the ToF data. Therefore, comparison of the overall attenuation with the effective attenuation yield the absorption coefficient μₐ of the superficial layer 20s.

As discussed, a secondary effect of the superficial layer 20s is to alter the shape of the early/rising portion of the ToF curve. In an embodiment, the processing circuit 130 is configured to determine the optical properties of the superficial layer 20s by fitting the measured ToF curve with a model ToF distribution derived from a theoretical multi-layer diffusive media model convolved with the IRF, as described above. The IRF is measured using the first reference optical path 141 as described above. This enable absorption and reduced scattering coefficients μₐ and μₛ′ of the superficial layer 20s to be extracted as well as absorption and reduced scattering coefficients of the bulk 20b of the target region 20.

ToF measurements at multiple wavelengths can help further quantify the superficial layer properties. In embodiments where the optical measurement system is configured to perform ToF measurements at a range of wavelengths, the processing circuit 130 may be configured to perform the above analysis and determine the presence of and/or one or more optical properties of a superficial layer 20s at each wavelength. In this case, the processing circuit 130 is configured to identify the superficial layer 20s based on a comparison of an optical property obtained at two or more wavelengths to spectral characteristics of known superficial layers 20s. For example, a superficial layer 20s may have a characteristic absorption or scattering spectrum, which enables it to be identified. Where there are multiple layers contributing to the superficial layer 20s (e.g. melanin, nail polish etc.), each may have a different characteristic absorption/scattering spectrum. As such, in one embodiment, the processing is circuit 130 is figured to identify the superficial layer 20 based on a comparison of the measured optical properties with one or more stored superficial layer absorption and/or scattering spectra.

As a specific example, melanin absorbs more at 660 nm than at 940 nm. Melanin provides skin pigmentation, so if the effective attenuation is closer to the overall (CW) attenuation at 940 nm than at 660 nm, this suggests dark pigmentation due to melanin. However, for lightly pigmented skin, the difference in absorption at 660 nm and 940 nm is relatively small due to the lower content of melanin. As such, the device 10 can be used to predict skin pigmentation, which can be used to compensate or calibrate CW measurements, as described above.

In an embodiment, the processing circuit 130 is configured to determine an optical biomarker value of the superficial layer 20s based, at least in part, on the determined one or more optical properties of the superficial layer. The optical biomarker value of the superficial layer may be or comprise a superficial layer identifier and/or index and/or a skin pigment index. For example, the superficial layer index may indicate a skin colour or level of pigmentation. The superficial layer identifier may indicate what the superficial layer is, e.g. skin pigmentation, scare tissue, nail, nail polish, collagen, layer of blood etc.

Movement is one of the key sources of errors in wearable biophonic devices, such as fitness trackers, pulse oximeters, smart watches etc. In an embodiment, the processing circuit 130 is configured to measure a series of ToF distributions over a period of time using a high count rate SPAD array 121a (described above), and detect a motion artefact based on dynamic changes in the shape and/or attenuation of the ToF distributions. Information on a motion artefacts derived from ToF measurements may also be used to compensate for motion artefacts in CW measurements the optical biomarkers, as described above.

Motion artefacts may arise due to motion of the device 10 relative to the target region 20, and/or motion of tissue e.g. expansion and contraction. Different types of motion cause have different effects on the ToF data.

Motion of the tissue during exercise causes expansion and contraction of the superficial layer 20s. Such motion artefacts can therefore be detected based on dynamic changes in the determined one or more optical properties of the superficial layer 20s (as described above).

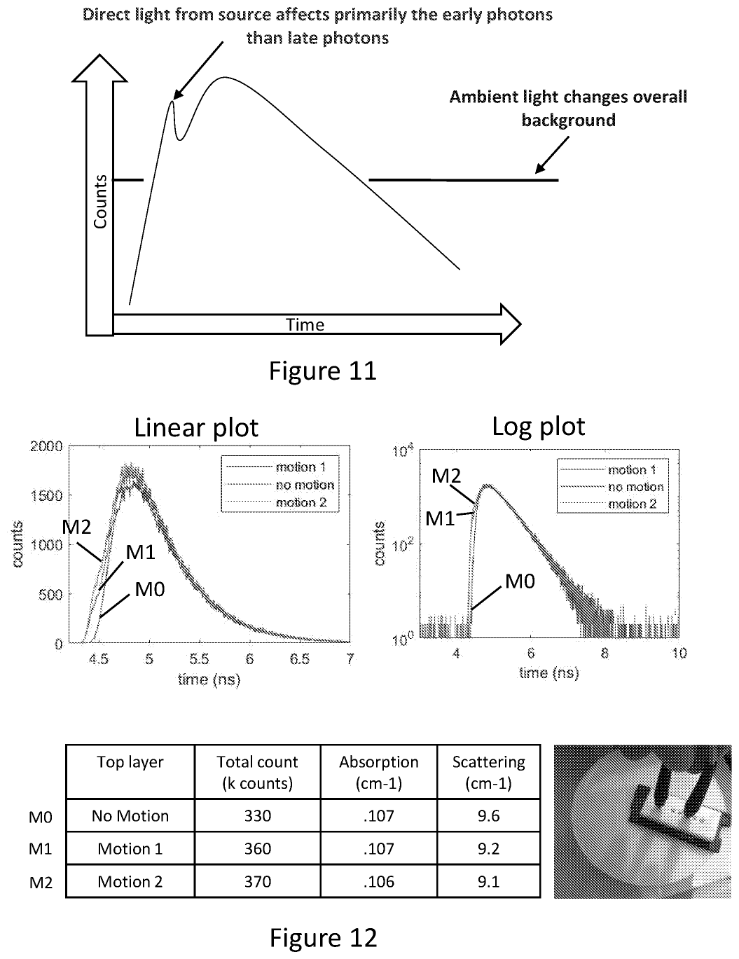

Movement of the device 10 relative to the target region 20 causes transient decoupling of the light beam from the target region which can allow direct light from the source and/or ambient light to reach the photodetector 121. Direct light from the source primarily effects the early photon pattern/rising portion of the ToF curve, whereas ambient light effects the background signal, as illustrated in FIG. 11. The effect of ambient light on ToF measurements is substantially reduced by the use of a high-count rate SPAD array 121a and by using appropriate optical filters 123 at the detection system 120 to allow only the wavelength of interest to reach the photodetector 121a. Motion artifacts from direct light can be detected by monitoring changes in the early photon patterns using the high-count rate SPAD array 121a. This can then be compensated for in the optical property extraction, as described below. This is a significant advantage over conventional CW-based devices which cannot compensate for direct light artefacts.

FIG. 12 shows experimental ToF data obtained from direct ToF measurements of a tissue mimicking phantom in a lab set up demonstrating the effects of different types of motion on the early photon pattern. Motion is simulated by lifting the source and detector fibres from the surface of the phantom (see upper right image). Curve M0 shows the ToF data for no motion, whereas curves M1 and M2 show ToF data for different motion. The table shows the results of fitting a single layer model ToF distribution to the whole ToF curve. As seen, the motion does not significantly impact the extracted absorption properties, as these are primarily determined by the tail of the ToF data, whereas the extracted scattering properties, which are primarily determined by the rise of the ToF data, show significant deviation with motion.

In an embodiment, the processing circuit 130 is configured to detect a motion artefact based on a comparison of the shape of the rising portion of the measured ToF distribution with a model ToF distribution derived from a theoretical single or multilayer model convolved with the IRF. The motion artefact may be detected based on a predefined divergence from the expected shape of the rising portion of the ToF distribution, such as an anomalous peak, dip and/or curvature. In response to detecting a motion artefact, the processing circuit 130 can continue to extract optical properties by fitting a model ToF distribution to the tail of the ToF curve. This may be an effective approach for deep tissue biomarker assessment which is most sensitive to late photon or the tail of the ToF curve.

Alternatively, and particular for stronger movements, analysis of the early photon pattern can allow estimation of the changing source illumination pattern, e.g. a change in spot size or shape. This can be fed into the theoretical model to correct the model ToF curve and improve the accuracy of the biomarker estimates. By simulating various kinds of reflection during controlled motion of the device 10, a database of these patterns (i.e. shape of the rising portion) can be created. In this case, in response to detecting a motion artefact, the processing circuit 130 is configured to compare the shape of the rising portion of the ToF distribution with the database of simulated motion artefacts, apply a correction to the model ToF distribution based on a selected one of the simulated motion artefacts, and extract the one or more optical properties of the target region 20 from the shape of the ToF distribution using the corrected model ToF distribution. In this way, motion artefacts can be effectively detected and compensated for in real time, reducing any errors in the extracted optical properties.

Motion artefacts in CW-measurements typically add noise to measured photoplethysmography (PPG) signals which follow the heart rate, i.e. in the frequency range 1 to 3 Hz (higher rate during intense cardio exercise. The movement of superficial layer 20s tissue may cause changes in the PPG signal due to compression of tissue, which adds noise in CW PPG signals. However, this is not such a problem in ToF data as the shape of the ToF distribution is largely insensitive to superficial layer 20s properties, whether static or dynamic. As such, to resolve such artefacts, and/or to resolve early and late systolic peak, dicrotic notch, depending on the application, a sampling frequency of at least few times (2-10) the highest frequency of interest (e.g. 3 Hz) is needed to satisfy the Nyquist criteria Slower motion artefacts, e.g. due to change in body position during sleep, can be monitored slowly in the sub Hz (0.01-1 Hz) range.

The motion artefact itself, e.g. detection of changes in dynamics of superficial layer, and/or early photons) can be used as optical biomarker or physiological monitor of the target region or human body or behaviour.

This project has received funding from the European Union's Horizon 2020 research and innovation programme under grant agreement No 101016087.

From reading the present disclosure, other variations and modifications will be apparent to the skilled person. Such variations and modifications may involve equivalent and other features which are already known in the art, and which may be used instead of, or in addition to, features already described herein.

Features which are described in the context of separate embodiments may also be provided in combination in a single embodiment. Conversely, various features which are, for brevity, described in the context of a single embodiment, may also be provided separately or in any suitable sub-combination.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

1. A wearable device configured to perform standardised time domain diffuse optical spectroscopy, comprising:
an optical measurement system including an illumination system with a light source configured to illuminate a target region of the body, at an injection point, with a pulsed or modulated beam of light thereby producing scattered light from the interaction of the light beam with the target region, a photodetection system configured to detect scattered light exiting the target region at a distance from the injection point and provide a measurement signal, and a processing circuit in communication with the illumination system and the photodetection system for time of flight (ToF) data acquisition, wherein the optical measurement system comprises a first reference optical path between the light source and photodetection system to couple at least a portion of the beam to the photodetection system to provide a first reference signal, wherein the first reference optical path comprises a tissue mimicking phantom with one or more known optical properties, wherein the processing circuit is configured to:
measure a ToF distribution for photons detected in response to the light beam being directed towards the target region;
determine one or more optical properties of the target region from measured ToF distribution;
measure a ToF distribution for photons detected in response to the at least a portion of the light beam being directed towards the phantom along the first reference optical path;
determine one or more calibration factors for use in determining the one or more optical properties of the target region; and
determine one or more optical biomarker values based, at least in part, on the one or more optical properties of the target region.

2. The wearable device of claim 1, wherein the processing circuit is configured to:
extract one or more optical properties of the phantom from the shape of the measured ToF distribution; and
determine, based on a comparison of the extracted one or more optical properties with the known optical properties of the phantom, a first calibration factor to apply to one or more optical properties extracted from the shape of the ToF distribution measured from the target region; and optionally or preferably,
wherein the first reference optical path comprises a first optical waveguide, fibre or light pipe for guiding the light pulses.

3. The wearable device of claim 1, wherein the processing circuit is further configured to:
extract the instrument response function (IRF) from the measured ToF distribution of photons detected from the phantom based at least in part on the known optical properties of the phantom;
store the extracted IRF; and optionally or preferably,
extract the one or more optical properties of the phantom from the shape of the measured ToF distribution by comparison to a model ToF curve derived from a theoretical diffusive media model convolved with the stored IRF.

4. The wearable device of claim 1, wherein the optical measurement system is configured to operate in a measurement mode in which the ToF distribution of photons detected from the target region is measured, and a first reference mode in which the ToF distribution of photons detected from the phantom is measured; and optionally or preferably wherein the optical measurement system comprises a first optical element operable to selectively couple the at least a portion of the light beam to the photodetection system via the first reference optical path to measure a ToF distribution of photons detected from the phantom in the first reference mode; and further optionally or preferably,
wherein the first optical element is operable to selectively block, transmit or direct the at least a portion of the beam along the first reference optical path.

5. (canceled)

6. The wearable device of claim 1, wherein the optical measurement system comprises a second reference optical path between the illumination system and photodetection system to couple at least a portion of the light beam directly to the photodetection system and provide a second reference signal, and wherein the processing circuit is further configured to:
measure the IRF for photons detected in response to the at least a portion of the light beam being directed towards the photodetection system along the second reference optical path; and
store the measured IRF; and optionally or preferably,
wherein the second reference optical path comprises a second optical waveguide, fibre or light pipe for guiding the light pulses.

7. The wearable device of claim 3, wherein the processing circuit is further configured to:
extract the one or more optical properties of the target region from the shape of the measured ToF distribution by comparison to a model ToF curve derived from a theoretical diffusive media model convolved with the stored IRF; and/or
determine, based on the stored IRF, a second calibration factor to apply to the ToF distribution to correct for drift in the optical measurement system for use in determining an overall attenuation of light by the target region.

8. The wearable device of claim 6, wherein the optical measurement system is configured to operate in a measurement mode in which the ToF distribution of photons detected from the target region is measured, and a second reference mode in which the IRF is measured; and optionally or preferably, wherein the optical measurement system comprises a second optical element operable to selectively couple the at least a portion of the beam to the photodetector via the second reference optical path to measure the IRF in the second reference mode; and further optionally or preferably, wherein the second optical element is operable to selectively block, transmit or direct the at least a portion of the beam along the second reference optical path.

9. The wearable device of claim 1, wherein the first reference optical path comprises a delay line to temporally separate the first reference signal from the measurement signal and permit simultaneous measurement of the ToF distribution for the phantom and the ToF distribution for the target region; and/or
the wearable device of claim 6, wherein the second reference optical path comprises a delay line to temporally separate the second reference signal from the measurement signal and permit simultaneous measurement of the IRF and the ToF distribution of photons detected from the target region.

10. The wearable device of claim 1, wherein the optical measurement system is configured to perform ToF measurements and continuous wave (CW) measurements of scattered light, wherein the optical measurement system comprises a ToF measurement mode for ToF measurements of scattered light, and one of a hardware CW measurement mode for CW measurements of scattered light and a software CW measurement mode in which the processing circuit is configured extract a CW measurement signal representing the amount of scattered light from the measured ToF distribution by summing all or a part of the measured ToF distribution,
wherein, in the software or hardware CW measurement mode, the processing circuit is configured to:
measure a timecourse of the amount of scattered light detected from the target region in response to the continuous beam of light being directed to the target region;
determine one or more optical biomarker values based, at least in part, on an optical property of the target region extracted from the measured timecourse; and
apply a correction factor to the measured timecourse and/or the one or more optical biomarker values based, at least in part, on one or more static and/or dynamic optical properties or optical biomarker values determined in the ToF measurement mode, optionally or preferably, derived from an optical biomarker value of a superficial layer of the target region determined in the ToF measurement mode.

11. The wearable device of claim 1, wherein the processing circuit is configured to determine the presence of and/or one or more optical properties of a superficial layer of the target region based on the attenuation and/or shape of the measured ToF distribution; and optionally or preferably based on a comparison to a model ToF curve derived from a theoretical multi-layer diffusive media model convolved with the IRF.

12. The wearable device of claim 11, wherein the processing circuit is configured to:
determine an overall attenuation of photons by the target region based on a comparison of the integrated photon count of the measured ToF distribution and the output of the light source;
determine an effective attenuation of photons by the target region based on one or more optical properties of the target region extracted from the shape of the measured ToF distribution; and
determine the presence of and/or the one or more optical properties of a superficial layer of the target region based on a comparison of the overall attenuation with the effective attenuation; and, optionally or preferably,
wherein one or more optical properties of the target region are extracted from the shape of the measured ToF distribution based on a comparison to a model ToF curve derived from a theoretical diffusive media model convolved with the IRF.

13. (canceled)

14. The wearable device of claim 11, wherein the light source is configured to provide a pulsed or modulated beam of light at a plurality of different wavelengths, and the processing circuit is configured to:
control the light source to illuminate the target region with a pulsed or modulated beam of light at a plurality of different wavelengths;
measure, for each respective wavelength, a ToF distribution of detected photons; and
determine the presence of and/or one or more static or dynamic optical properties of a superficial layer of the target region based on analysis of the attenuation and/or shape of the measured ToF distribution at each wavelength; and, optionally or preferably, further based on a comparison with one or more stored superficial layer absorption spectra.

15. The wearable device of claim 11, wherein the processing circuit is configured to determine an optical biomarker value of the superficial layer based, at least in part, on the determined one or more optical properties of the superficial layer; and optionally or preferably,
wherein the optical biomarker value of the superficial layer is or comprises a superficial layer identifier and/or a skin pigment index.

16. The wearable device of claim 10, wherein:
the processing circuit is configured to determine the presence of and/or one or more optical properties of a superficial layer of the target region based on the attenuation and/or shape of the measured ToF distribution;
the processing circuit is configured to determine an optical biomarker value of the superficial layer based, at least in part, on the determined one or more optical properties of the superficial layer; and
the correction factor is derived from an optical biomarker value of the superficial layer; and optionally or preferably,
wherein the optical biomarker value of the superficial layer is or comprises a superficial layer identifier and/or a skin pigment index.

17. The wearable device of claim 1, wherein the processing circuit is configured to
measure a series of ToF distributions over a period of time to monitor the one or more optical biomarker values; and
detect a motion artefact in at least one of the ToF distributions based on dynamic changes in the shape and/or attenuation of the ToF distributions; and optionally or preferably wherein the processing circuit is configured to:
determine, for each ToF distribution in the time series, one or more optical properties of a superficial layer of the target region based on an analysis of the attenuation and/or shape of the ToF distribution; and
detect a motion artefact in at least one of the ToF distributions based on dynamic changes in the determined one or more optical properties of the superficial layer.

18. (canceled)

19. The wearable device of claim 17, wherein the processing circuit is configured to:
detect a motion artefact in at least one of the ToF distribution based on a comparison of the shape of the rising portion of the measured ToF distribution with a model ToF distribution derived from a theoretical diffusive media model convolved with the IRF; and
in response to detecting a motion artefact:
extract the one or more optical properties of the target region from the shape of the tail portion of the ToF distribution; and/or
compare the shape of the rising portion of the ToF distribution with a database of simulated motion artefacts, apply a correction to the model ToF curve based on a selected one of the simulated motion artefacts, and extract the one or more optical properties of the target region from the shape of the ToF distribution using the corrected model ToF distribution.

20. The wearable device of claim 1, wherein the one or more optical biomarkers are selected from the group comprising: tissue oxygen saturation, arterial oxygen saturation, oxy-haemoglobin, deoxy-haemoglobin, lipid, water, collagen, hydration, glucose, melanin, thyrosine, thyroglobulin, cytochrome c-oxidise, carboxy-haemoglobin, methe-haemoglobin.

21. The wearable device of claim 1, wherein the photodetection system comprises an array of single photon detectors and wherein the processing circuit comprises a plurality of time-to-digital converters (TDC), each TDC coupled to a respective one or group of single photon detectors in the array, wherein the array of single photon detectors is divided into two or more segments each comprising a subgroup of single photon detectors and the outputs of the or each TDC associated with a respective segment or subgroup are coupled together,
wherein the optical measurement system is configured to couple scattered light exiting the target region to a first segment of the array to provide the measurement signal, and to couple the at least a portion of the light beam directed along the first reference optical path comprising the tissue mimicking phantom to a second segment of the array to provide the first reference signal, and
wherein the processing circuit is configured to:
determine a ToF of photons detected at each single photon detector in a respective segment based, at least in part, on the coupled output of the or each TDC associated with that segment; and
generate, for each segment, a ToF distribution of the sum of photons detected by the subgroup of single photon detectors in the respective segment accumulated over a plurality of light pulses; and optionally or preferably
wherein the optical measurement system is configured to measure the measurement signal and first reference signal simultaneously; and further optionally or preferably,
wherein the processing circuit is configured to determine a ToF of photons by time correlated single photon counting, or by time gated single photon detection; and further optionally or preferably,
wherein the optical measurement system is configured to provide a photon count rate of greater than 100 Mcps without pile up.

22. The wearable device of claim 21, wherein the optical measurement system comprises a second reference optical path between the illumination system and photodetection system to couple at least a portion of the light beam directly to the photodetection system and provide a second reference signal, and wherein the processing circuit is further configured to:
measure the IRF for photons detected in response to the at least a portion of the light beam being directed towards the photodetection system along the second reference optical path; and
store the measured IRF; and
wherein the optical measurement system is configured to couple the at least a portion of the light beam directed along the second reference optical path to a third segment of the array to provide the second reference signal, optionally or preferably,
wherein the optical measurement system is configured to measure the measurement signal and first and/or second reference signal simultaneously.

23. The wearable device of claim 1, wherein the wearable device is or comprises a pulse oximeter, and the determined one or more optical biomarker values include tissue oxygen saturation, arterial oxygen saturation, oxy-haemoglobin, and deoxy-haemoglobin.

24. A method of performing standardised time domain diffuse optical spectroscopy in a wearable device comprising an optical measurement system configured to perform time domain diffuse optical spectroscopy, the optical measurement system comprising an illumination system and a photodetection system, the method comprising:
measuring a time of flight (ToF) distribution for photons detected in response to a pulsed or modulated beam of light being directed towards a target region of the body;
measuring the IRF in response to a pulsed or modulated beam of light being directed towards a photodetection system along a reference optical path without interacting with the target region;
extracting one or more optical properties of the target region from the shape of the measured ToF distribution by comparison to a model ToF curve derived from a theoretical diffusive media model convolved with the measured IRF; and/or
determining, based on the measured IRF, a calibration factor to apply to the ToF distribution to correct for drift in the optical measurement system.

25. The method of claim 24, further comprising:
measuring a ToF distribution for photons detected in response to a pulsed or modulated beam of light being directed towards a tissue mimicking phantom located in the same or a different reference optical path;
extracting one or more optical properties of the phantom from the shape of the measured ToF distribution by comparison to a model ToF curve derived from a theoretical diffusive media model convolved with the measured IRF; and
determining, based on a comparison of the extracted one or more optical properties with known optical properties of the phantom, a calibration factor to apply to the one or more optical properties extracted from the shape of the ToF distribution measured from the target region.

26. The method of claim 24, further comprising:
extracting a CW measurement signal from the measured ToF distribution from the target region by summing all or a part of the measured ToF distribution from the target region, the CW measurement signal representing an amount of scattered light detected;
measuring a series of ToF distributions over a period of time and extracting a timecourse of the CW measurement signal in response to a pulsed or modulated beam of light being directed to the target region;
determining one or more optical biomarker values based, at least in part, on an optical property of the target region extracted from the measured timecourse; and
applying a correction factor to the measured timecourse and/or the one or more optical biomarker values based, at least in part, on one or more static and/or dynamic optical properties extracted from the ToF measurements, optionally or preferably, wherein the correction factor is derived from an optical property of a superficial layer of the target region determined from the ToF measurements.

27. (canceled)

28. The method of claim 24, further comprising:
detecting the presence of and/or determining one or more optical properties of a superficial layer of the target region based on the attenuation and/or shape of the measured ToF distribution.

29. The method of claim 28, further comprising:
measuring a series of ToF distributions over a period of time; and
detecting a motion artefact in at least one of the ToF distributions based on dynamic changes in the determined one or more optical properties of the superficial layer, optically or preferably, based on dynamic changes in the shape and/or attenuation of the ToF distributions.

30. The method of claim 24, wherein the optical measurement system is further configured to perform continuous wave (CW) diffuse optical spectroscopy in a hardware CW mode, the method comprising:
operating the optical measurement system in a CW mode;
measuring a timecourse of the amount of scattered light detected from the target region in response to a continuous beam of light being directed to the target region;
determining one or more optical biomarker values based, at least in part, on an optical property of the target region extracted from the measured timecourse; and
applying a correction factor to the one or more optical biomarker values based, at least in part, on one or more static and/or dynamic optical properties determined in the ToF measurement mode, optionally or preferably, derived from an optical property of a superficial layer of the target region determined in the ToF measurement mode.

31. A method of performing standardised time domain diffuse optical spectroscopy in a wearable device, comprising:
performing time of flight (ToF) measurements of photons detected in response to a pulsed or modulated beam of light being directed towards a target region of the body using a high count rate photodetection system to generate ToF data;
extracting one or more optical properties of the target region from the ToF data;
summing all or some of the photons detected in a measurement period to extract a high count rate photoplethysmography (PPG) signal;
extracting one or more optical biomarker values from the PPG signal; and
extracting one or more optical properties of a superficial layer of the target region based on the shape and/or attenuation of the ToF data, and
correcting the one or more optical biomarker values extracted from the PPG signal based on the one or more optical properties of the superficial layer extracted from the ToF data.

32. The method of claim 31, further comprising detecting a motion artefact in the ToF data based on dynamic changes in the shape and/or attenuation of the ToF data, and processing the PPG signal to remove the detected artefact.

33. The method of claim 31, further comprising determining one or optical biomarker values based on the extracted optical properties of the target region selected from the group of: tissue oxygen saturation, arterial oxygen saturation, oxy-haemoglobin, deoxy-haemoglobin, lipid, water, collagen, hydration, glucose, melanin, thyrosine, thyroglobulin, and cytochrome c-oxidise, carboxy-haemoglobin, methe-haemoglobin; and/or, wherein the one or more optical biomarker values extracted from the PPG signal are selected from the group of: arterial tissue oxygenation, heart rate, and respiration rate.

34. The method of claim 31, wherein performing ToF measurements comprises:
performing indirect ToF measurements using a modulated beam of light, optionally using a spread spectrum method; and/or performing ToF measurements at a plurality of wavelengths.

35-36. (canceled)

37. A method of performing standardised diffuse optical spectroscopy in a wearable device comprising:
performing time of flight (ToF) measurements of photons detected in response to a pulsed or modulated beam of light being directed towards a target region of the body to generate ToF data;
extracting one or more optical properties of the target region from the ToF data;
measuring a timecourse of the amount of scattered light detected from the target region in response to a continuous beam of light being directed to the target region to generate CW data;
processing the measured CW data to filter out or remove motion artefacts and/or physiological effect based, at least in part, on one or more static and/or dynamic optical properties of the target region extracted from the ToF data; and
determining one or more optical biomarker values based, at least in part, on an optical property of the target region extracted from the processed CW data.
